(19) United States
(12) Patent Application Publication
(10) Pub. No.: US 2026/0286222 A1
(43) Pub. Date: Sep. 24, 2026

(54) CHIRAL PHOTOSENSITIVE MOLECULAR SWITCH, CHOLESTERIC LIQUID CRYSTAL COMPOSITION, DISPLAY PANEL AND PREPARATION METHOD THEREOF

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Xiaojuan Wu, Beijing (CN); Jinbao Guo, Beijing (CN); Jiaxing Wang, Beijing (CN); Danxing Hou, Beijing (CN); Cuiyu Chen, Beijing (CN); Zhiqiang Yu, Beijing (CN); Dawei Feng, Beijing (CN); Feng Liu, Beijing (CN); Jinshuai Duan, Beijing (CN); Ning Wang, Beijing (CN); Yichi Zhang, Beijing (CN)

(21) Appl. No.: 19/475,406

(22) PCT Filed: Oct. 23, 2023
(86) PCT No.: PCT/CN2023/125940

§ 371 Date: Oct. 14, 2025

(51) Int. Cl. C09K 19/34; C09K 19/02; C09K 19/58

(52) U.S. Cl. CPC C09K 19/3491; C09K 19/02; C09K 19/586

(57) ABSTRACT

Provided are a chiral photosensitive molecular switch, a cholesteric liquid crystal composition, a display panel, and a preparation method thereof. The chiral photosensitive molecular switch has two stable states, and may switch, in response to light irradiation, from one stable state to the other stable state which may stably exist. The cholesteric liquid crystal composition includes: a chiral photosensitive molecular switch, a bent-core molecular mixture, a rod-like single crystal mixture and a non-photosensitive chiral dopant. The cholesteric liquid crystal composition may be used to prepare a cholesteric liquid crystal display panel, where the cholesteric liquid crystal has two stable states with different handedness and may reflect light of different handedness.

### CROSS REFERENCE TO RELATED APPLICATION(S)

This application is a National Stage Application of International Application No. PCT/CN2023/125940, filed on Oct. 23, 2023, entitled “CHIRAL PHOTOSENSITIVE MOLECULAR SWITCH, CHOLESTERIC LIQUID CRYSTAL COMPOSITION, DISPLAY PANEL AND PREPARATION METHOD THEREOF”, the entire content of which is incorporated herein in its entirety by reference.

### TECHNICAL FIELD

The present disclosure relates to a field of display technology, in particular to a chiral photosensitive molecular switch, a cholesteric liquid crystal composition, a display panel and a preparation method thereof.

### BACKGROUND

Cholesteric liquid crystals have a special helical structure and are extremely sensitive to various external stimuli (an electric field, a magnetic field, light, temperature, humidity, pH, a mechanical force), thus having broad application prospects in fields such as dynamic displays, sensors, and tunable optical lasers, and attracting extensive attention from researchers. Oblique heliconical cholesteric (ChOH) liquid crystals, as a special type of cholesteric liquid crystals, have a pitch that decreases with increasing electric field intensity within a certain range, enabling selective reflection over a broad spectral range from ultraviolet to near-infrared, which makes oblique heliconical cholesteric liquid crystal materials show great application potential in fields such as full-color reflective displays, smart windows, tunable filters, and holography. However, due to the special molecular arrangement, the reflectivity of the oblique heliconical cholesteric liquid crystal materials under normal viewing angles when regulated by an electric field is relatively low, and accordingly, the brightness of liquid crystal devices is low, which limits the practical application of the oblique heliconical cholesteric liquid crystal materials to a certain extent.

How to optimize the oblique heliconical cholesteric liquid crystal materials to ensure that oblique heliconical cholesteric liquid crystal materials have high reflectivity, high contrast, and good stability is one of the important research topics for researchers.

The above information disclosed in this section is only for understanding of the background of the inventive concept of the present disclosure. Therefore, the above information may contain information that does not constitute the related art.

### SUMMARY

In an aspect, a chiral photosensitive molecular switch is provided, where a chemical structure of the chiral photosensitive molecular switch is represented by the following general formula I:

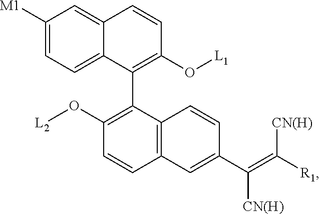

where M1 is selected from groups with structures as shown in

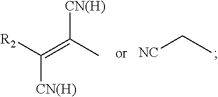

groups R₁ and R₂ are the same or different, each including a benzene ring, biphenyl, an aromatic ring or an aromatic heterocyclic ring; and L₁ and L₂ each includes a C₁-C₁₀ alkyl group.

According to some exemplary embodiments, a chemical structure of at least one of the groups R₁ and R₂ is represented by one of the following formulas:

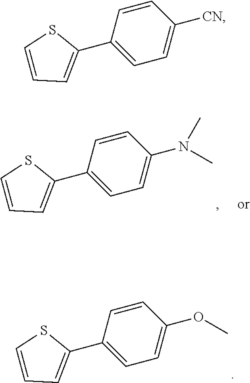

According to some exemplary embodiments, the chemical structure of the chiral photosensitive molecular switch is represented by one of the following formulas:

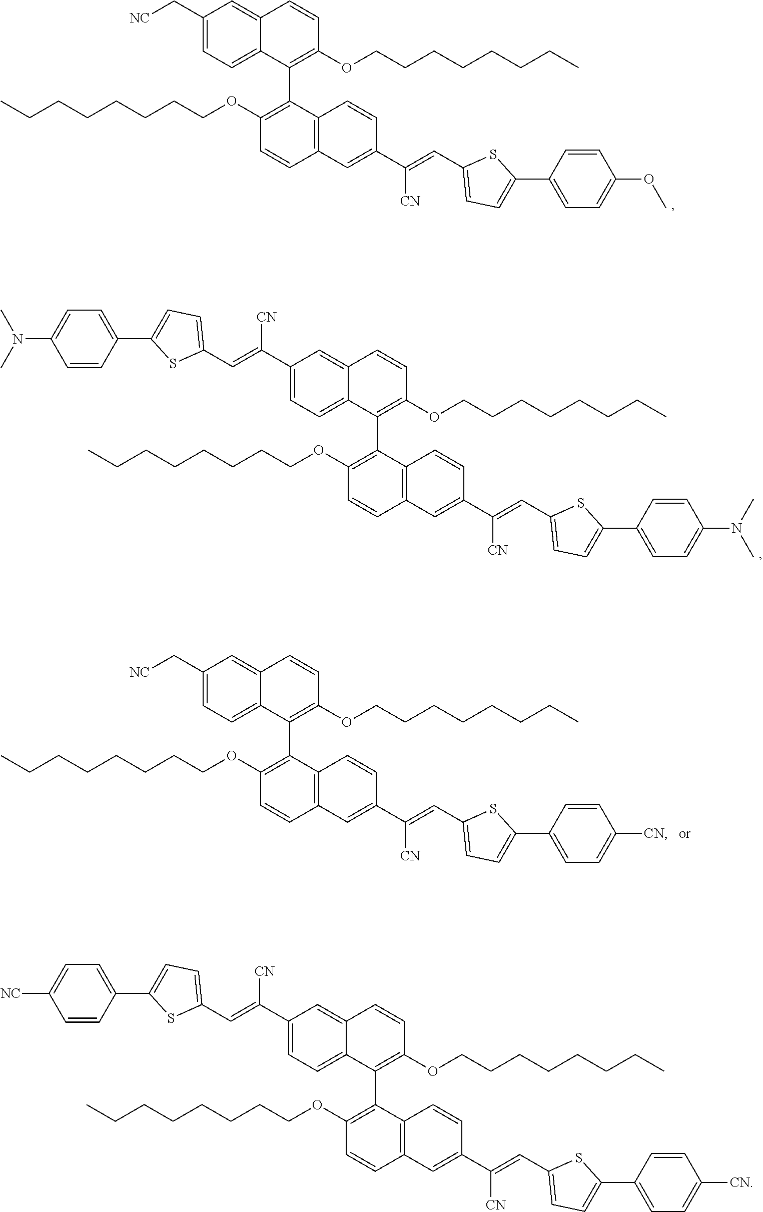

In another aspect, a cholesteric liquid crystal composition is provided, where the composition includes the chiral photosensitive molecular switch according to any one of the above items.

According to some exemplary embodiments, the composition further includes a bent-core molecular mixture, a rod-like single crystal mixture and a non-photosensitive chiral dopant.

According to some exemplary embodiments, a proportion of the chiral photosensitive molecular switch is 0.5% to 10% by weight of the cholesteric liquid crystal composition.

According to some exemplary embodiments, a proportion of the non-photosensitive chiral dopant is 0.5% to 10% by weight of the cholesteric liquid crystal composition.

According to some exemplary embodiments, a proportion of the bent-core molecular mixture is 30% to 70% by weight of the cholesteric liquid crystal composition, and/or a proportion of the rod-like single crystal mixture is 30% to 70% by weight of the cholesteric liquid crystal composition.

According to some exemplary embodiments, by weight of the cholesteric liquid crystal composition, a proportion of the bent-core molecular mixture is 30% to 70%, a proportion of the rod-like single crystal mixture is 30% to 70%, a proportion of the chiral photosensitive molecular switch is 5% to 10%, a proportion of the non-photosensitive chiral dopant is 5% to 10%, and a sum of the proportion of the bent-core molecular mixture, the proportion of the rod-like single crystal mixture, the proportion of the chiral photosensitive molecular switch and the proportion of the non-photosensitive chiral dopant is 100%.

In yet another aspect, a cholesteric liquid crystal display panel is provided, where the display panel includes: a first substrate and a second substrate arranged opposite to each other; and a cholesteric liquid crystal layer located between the first substrate and the second substrate, where the cholesteric liquid crystal layer includes a cholesteric liquid crystal composition, and the cholesteric liquid crystal composition includes the chiral photosensitive molecular switch according to any one of the above items, or the cholesteric liquid crystal composition is the composition according to any one of the above items.

According to some exemplary embodiments, the cholesteric liquid crystal layer includes a first cholesteric liquid crystal unit and a second cholesteric liquid crystal unit, a liquid crystal in the first cholesteric liquid crystal unit has an oblique heliconical structure with a first handedness, a liquid crystal in the second cholesteric liquid crystal unit has an oblique heliconical structure with a second handedness, and the first handedness is opposite to the second handedness.

According to some exemplary embodiments, an absolute value of a helical twisting power of the liquid crystal in the first cholesteric liquid crystal unit is substantially equal to an absolute value of a helical twisting power of the liquid crystal in the second cholesteric liquid crystal unit.

According to some exemplary embodiments, the display panel further includes: a first electrode located on the first substrate and a second electrode located on the second substrate; the cholesteric liquid crystal layer is configured such that a wavelength corresponding to a reflection peak of the cholesteric liquid crystal layer changes in response to a change in a voltage applied between the first electrode and the second electrode.

According to some exemplary embodiments, the cholesteric liquid crystal layer is configured such that: in response to a first voltage applied between the first electrode and the second electrode, the reflection peak of the cholesteric liquid crystal layer corresponds to a first wavelength; in response to a second voltage applied between the first electrode and the second electrode, the reflection peak of the cholesteric liquid crystal layer corresponds to a second wavelength; and in response to a third voltage applied between the first electrode and the second electrode, the reflection peak of the cholesteric liquid crystal layer corresponds to a third wavelength, where the first voltage, the second voltage and the third voltage are different from each other, and the first wavelength, the second wavelength and the third wavelength correspond to visible light of different colors respectively.

According to some exemplary embodiments, the cholesteric liquid crystal layer is configured such that the wavelength corresponding to the reflection peak of the cholesteric liquid crystal layer increases in response to a decrease in the voltage applied between the first electrode and the second electrode.

According to some exemplary embodiments, the first voltage is greater than the second voltage, and the second voltage is greater than the third voltage; the first wavelength is less than the second wavelength, and the second wavelength is less than the third wavelength.

According to some exemplary embodiments, the first substrate and the second substrate are cell-assembled to form an accommodating space, the cholesteric liquid crystal layer is located in the accommodating space, and orthographic projections of the first cholesteric liquid crystal unit and the second cholesteric liquid crystal unit on the first substrate are distributed alternately.

According to some exemplary embodiments, the display panel further includes a third substrate located between the first substrate and the second substrate; the cholesteric liquid crystal layer includes: a first sub-cholesteric liquid crystal layer located between the first substrate and the third substrate, and a second sub-cholesteric liquid crystal layer located between the third substrate and the second substrate; and the first sub-cholesteric liquid crystal layer includes the first cholesteric liquid crystal unit, and the second sub-cholesteric liquid crystal layer includes the second cholesteric liquid crystal unit.

In still another aspect, a method of preparing a cholesteric liquid crystal display panel is provided, including: providing a first substrate and a second substrate; forming a cholesteric liquid crystal layer between the first substrate and the second substrate, where the cholesteric liquid crystal layer includes a cholesteric liquid crystal composition, and the cholesteric liquid crystal composition includes the chiral photosensitive molecular switch according to any one of the above items, or the cholesteric liquid crystal composition is the composition according to any one of the above items.

According to some exemplary embodiments, the forming a cholesteric liquid crystal layer between the first substrate and the second substrate includes: injecting the cholesteric liquid crystal composition into an accommodating space formed between the first substrate and the second substrate; irradiating the cholesteric liquid crystal composition with light of a predetermined wavelength to form a first cholesteric liquid crystal unit and a second cholesteric liquid crystal unit in the cholesteric liquid crystal layer, where a liquid crystal in the first cholesteric liquid crystal unit has an oblique heliconical structure with a first handedness, a liquid crystal in the second cholesteric liquid crystal unit has an oblique heliconical structure with a second handedness, and the first handedness is opposite to the second handedness.

According to some exemplary embodiments, the forming a cholesteric liquid crystal layer between the first substrate and the second substrate includes: injecting the cholesteric liquid crystal composition into an accommodating space formed between the first substrate and the second substrate; irradiating the cholesteric liquid crystal composition with a mask and light of a predetermined wavelength, where a handedness of the liquid crystal in the cholesteric liquid crystal layer not exposed to the irradiation of the light of the predetermined wavelength remains unchanged to form a first cholesteric liquid crystal unit, and a handedness of the liquid crystal in the cholesteric liquid crystal layer exposed to the irradiation of the light of the predetermined wavelength is inversed to form a second cholesteric liquid crystal unit.

According to some exemplary embodiments, the forming a cholesteric liquid crystal layer between the first substrate and the second substrate includes: injecting the cholesteric liquid crystal composition into an accommodating space formed between the first substrate and a third substrate to form a first sub-cholesteric liquid crystal layer; injecting the cholesteric liquid crystal composition into an accommodating space formed between the third substrate and the second substrate to form a second sub-cholesteric liquid crystal layer; and irradiating the second sub-cholesteric liquid crystal layer with light of a predetermined wavelength, where a liquid crystal in the second sub-cholesteric liquid crystal layer is exposed to the irradiation of the light of the predetermined wavelength and undergoes handedness inversion to form a second cholesteric liquid crystal unit, and a liquid crystal in the first sub-cholesteric liquid crystal layer is not exposed to the irradiation of the light of the predetermined wavelength and remains handedness unchanged to form a first cholesteric liquid crystal unit.

### BRIEF DESCRIPTION OF THE DRAWINGS

Other objectives and advantages of the present disclosure will be more apparent through the following description of the present disclosure with reference to the accompanying drawings, which may assist in a comprehensive understanding of the present disclosure.

FIG. 1 shows a chemical structural formula of a chiral photosensitive molecular switch according to some embodiments of the present disclosure;

FIG. 2A and FIG. 2B respectively show schematic diagrams of two stable-state structures of oblique heliconical cholesteric liquid crystals according to some embodiments of the present disclosure;

FIG. 3 shows a schematic diagram of a preparation process of a chiral photosensitive molecular switch according to some embodiments of the present disclosure;

FIG. 4 shows a schematic structural diagram of a cholesteric liquid crystal display panel according to some embodiments of the present disclosure;

FIG. 5 shows a schematic diagram of a comparison between structures of a cholesteric liquid crystal display panel before and after light irradiation according to some embodiments of the present disclosure;

FIG. 6 shows a schematic structural diagram of a cholesteric liquid crystal display panel according to some embodiments of the present disclosure;

FIG. 7 shows a schematic diagram of a comparison between helical twisting powers of an oblique heliconical cholesteric liquid crystal system added with a chiral photosensitive molecular switch in different states according to some embodiments of the present disclosure;

FIG. 8 shows a schematic diagram of a relationship between a reflection peak of a cholesteric liquid crystal display panel and an applied voltage according to some embodiments of the present disclosure;

FIG. 9 shows a schematic diagram of a stacked structure of a cholesteric liquid crystal display panel according to some embodiments of the present disclosure;

FIG. 10 shows a flowchart of preparing a cholesteric liquid crystal display panel according to some embodiments of the present disclosure;

FIG. 11 shows a flowchart of forming a cholesteric liquid crystal layer between a first substrate and a second substrate in step S2 of FIG. 10;

FIG. 12 shows a schematic diagram of light irradiation in a preparation of step S22 according to some embodiments of the present disclosure; and

FIG. 13 shows a schematic diagram of light irradiation in a preparation of step S22 according to other embodiments of the present disclosure.

### DETAILED DESCRIPTION OF EMBODIMENTS

In order to make objectives, technical solutions and advantages of embodiments of the present disclosure more clear, technical solutions of the embodiments of the present disclosure will be clearly and completely described below with reference to the accompanying drawings. It is obvious that the embodiments described are only some embodiments of the present disclosure, rather than all embodiments. All other embodiments, which are derived by those of ordinary skill in the art from the embodiments of the present disclosure without carrying out inventive effort, fall within the protection scope of the present disclosure.

It should be noted that in the accompanying drawings, a size and relative size of elements may be exaggerated for purposes of clarity and/or description. As such, the size and relative size of various elements are not necessarily limited to those shown in the figures. In the description and the drawings, the same or similar reference signs denote the same or similar parts.

When an element is described as being “on”, “connected to” or “coupled to” another element, the element may be directly on, connected to or coupled to the another element or an intervening element may be present. However, when an element is described as being “directly on”, “directly connected to” or “directly coupled to” another element, there is no intervening element. Other terms and/or expressions used to describe a relationship between elements should be interpreted in a similar manner, such as, “between . . . and” versus “directly between . . . and”, “adjacent” versus “directly adjacent” or “on” versus “directly on”, etc. Moreover, a term “connection” may refer to a physical connection, an electrical connection, a communication connection, and/or a fluid connection. Furthermore, X, Y, and Z axes are not limited to three axes of a rectangular coordinate system, and may be interpreted in a broader sense. For example, X, Y, and Z axes may be perpendicular to each other, or may represent different directions that are not perpendicular to each other. For purposes of the present disclosure, “at least one of X, Y and Z” and “at least one selected from a group consisting of X, Y and Z” may be interpreted as X only, Y only, Z only, or any combination of two or more of X, Y and Z, such as XYZ, XYY, YZ and ZZ. As used herein, a term “and/or” includes any and all combinations of one or more of related items listed.

It should be noted that, although terms “first”, “second”, etc. may be used herein to describe various parts, components, elements, regions, layers and/or sections, these parts, components, elements, regions, layers and/or sections should not be limited by these terms. Rather, these terms are used to distinguish one part, component, element, region, layer or section from another. Thus, for example, a first part, a first component, a first element, a first region, a first layer, and/or a first section discussed below could be termed a second part, a second component, a second element, a second region, a second layer, and/or a second section without departing from teachings of the present disclosure.

For convenience in description, spatial relationship terms, such as “upper”, “lower”, “left”, “right” and the like, may be used herein to describe a relationship between one element or feature and another element or feature as illustrated in the figures. It will be understood that the spatial relationship terms are intended to encompass different orientations of a device in use or operation in addition to an orientation depicted in the figures. For example, if the device in the figures is turned over, elements described as “below” or “beneath” other elements or features would then be oriented “above” or “over” other elements or features.

As used herein, the terms “substantially”, “about”, “approximately”, “roughly”, and other similar terms are used as terms of approximation rather than as terms of degree, and are intended to account for inherent deviations in measured or calculated values that would be recognized by those of ordinary skill in the art. Taking into account factors such as process fluctuations, measurement problems, errors associated with measurement of particular quantities (i.e., limitations of a measurement system), etc., “about” or “approximately” as used herein includes the stated values, and indicates that the particular values as determined by those of ordinary skill in the art are within acceptable tolerances. For example, “about” may mean within one or more standard deviations, or within ±30%, ±20%, ±10% or ±5% of the stated values.

In a cholesteric liquid crystal material, a chiral additive is an indispensable part, which may induce a nematic phase to be converted into a cholesteric phase. In the chiral additive, there is a type of molecules with special properties, and the molecules have two stable states and may achieve mutual transformation between stable states under external stimuli, which may not only induce the transformation of nematic phase to cholesteric phase, making nematic molecules self-assemble into a helical structure, but also achieve the regulation of the pitch and reflection wavelength of cholesteric liquid crystals under external stimuli. The chiral additive with the special properties is called a chiral molecular switch. The chiral molecular switch may achieve the transformation of molecules between stable states under the action of external stimuli.

The chiral molecular switch must meet several conditions: 1. there are at least two stable states; 2. there is a switching method to enable molecules to transform from one stable state to another stable state; 3. there is a method to detect different stable states. According to different stimulation methods, molecular switches may be divided into: a light-controlled molecular switch, an electric-controlled molecular switch, a magnetic-controlled molecular switch, a pressure-controlled molecular switch, a heat-controlled electronic switch, and the like. Light control has attracted extensive attention from researchers due to its own advantages. At present, chiral molecules used to prepare light-responsive cholesteric liquid crystals mainly include azobenzene-based, diarylethene-based, molecular motor-based, and α-cyano-substituted diarylethene-based molecules, etc. The related light-responsive chiral molecules have their own characteristics. Azobenzene-based chiral molecules have a large change in helical twisting power, but the cis-isomer of azobenzene has poor thermal stability and is prone to thermal relaxation recovery; moreover, when regulating the pitch of cholesteric phase with chiral molecules prepared based on azobenzene groups, ultraviolet light must be used. However, ultraviolet light is harmful to organisms, which limits the practical application of light-responsive cholesteric liquid crystals. Diarylethene-based chiral molecules have good thermal stability, but the spatial configuration of diarylethene changes little before and after the isomerization reaction, thus in general, the change in helical twisting power of such chiral molecules is small. The helical twisting power of molecular motor-based chiral molecules may change greatly, but their light regulation process is often irreversible and may only recover through thermal relaxation. α-cyano-substituted diarylethene is a photosensitive molecule with a π-conjugated group; under the stimulation of a light source, α-cyano-substituted diarylethene may be subjected to Z/E isomerization; after being connected with a chiral unit, the helical twisting power of chiral photosensitive molecules may be changed, and cholesteric liquid crystals prepared by adding α-cyano-substituted diarylethene chiral photosensitive molecular switches may show good light-responsive properties to light sources in specific wavelength bands, ultimately causing the reflection color of cholesteric liquid crystals to change and showing good thermal stability.

Due to the unique optical activity, circular dichroism, and selective Bragg reflection of the cholesteric liquid crystals, the cholesteric liquid crystals have shown broad application prospects in optical materials. However, conventional cholesteric liquid crystals may only achieve the regulation of a single reflection color or the regulation of a scattering state of the cholesteric liquid crystals by adjusting the voltage. Compared with traditional cholesteric liquid crystals, an oblique heliconical cholesteric (ChOH) liquid crystal material has the greatest advantage in that its pitch, tilt angle, and molecular chirality may change under external stimuli such as electric fields, thereby achieving selective reflection over a broad spectral range from ultraviolet to near-infrared. Therefore, the oblique heliconical cholesteric liquid crystal material has more advantages than an ordinary cholesteric material in regulating the reflection color by an electric field. People may use a single oblique heliconical cholesteric liquid crystal device to achieve selective reflection of light from ultraviolet to visible light and then to infrared light by simply applying an external electric field.

However, in a natural light state, due to the characteristics of its own oblique heliconical structure, the oblique heliconical cholesteric liquid crystal material has a low reflectivity at a normal viewing angle, and accordingly, the display contrast of the obtained liquid crystal device is low, which affects the possibility of the practical application of the liquid crystal device to a certain extent. Therefore, it is necessary to find an oblique heliconical cholesteric liquid crystal material with high reflectivity and high contrast.

The inventors find in the research that, by adding a chiral photosensitive molecular switch to the oblique heliconical cholesteric liquid crystal material and adopting the strategy of regulating the handedness of oblique heliconical cholesteric liquid crystals with light and regulating the reflection band with an electric field, an oblique heliconical cholesteric liquid crystal device with high reflectivity, high contrast, and good stability, which may be applied to electric field-regulated reflection in the full spectral range, may be obtained.

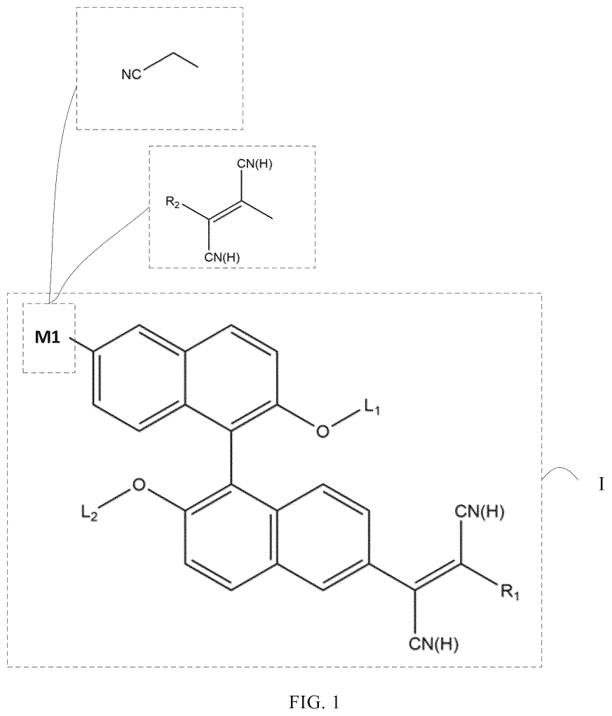

FIG. 1 shows a chemical structural formula of a chiral photosensitive molecular switch according to some embodiments of the present disclosure.

Some embodiments of the present disclosure provide at least one chiral photosensitive molecular switch. Referring to FIG. 1, the chemical structure of the chiral photosensitive molecular switch is represented by the following general formula I:

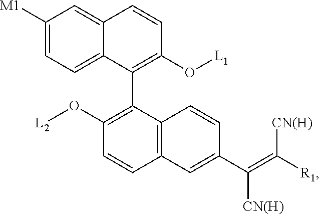

where M1 is selected from groups with structures as shown in

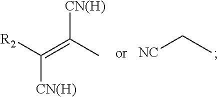

groups R₁ and R₂ are the same or different, each including a benzene ring, biphenyl, an aromatic ring or an aromatic heterocyclic ring; and L₁ and L₂ each includes a C₁-C₁₀ alkyl group.

It should be noted that in each general formula herein, the group CN(H) means that the substitution position of the —CN group may be on one C of a double bond or on the other C of the double bond. For example, in the general formula, the two groups CN(H) connected to both ends of a double bond indicate that the substitution position of the —CN group may be on either C of the double bond.

By adding a chiral photosensitive molecular switch to the oblique heliconical cholesteric liquid crystal, the oblique heliconical cholesteric liquid crystal may be regulated by both an electric field and light. The oblique heliconical cholesteric liquid crystal added with the chiral photosensitive molecular switch may transform from one stable state to another stable state under light regulation. For example, the oblique heliconical cholesteric liquid crystal has two stable states, and the two stable states include a left-handed oblique heliconical cholesteric phase and a right-handed oblique heliconical cholesteric phase. Assuming that the initial state of the oblique heliconical cholesteric liquid crystal is the right-handed oblique heliconical cholesteric phase, after irradiation with light of a specific wavelength, the oblique heliconical cholesteric liquid crystal may transform into the left-handed oblique heliconical cholesteric phase. By designing the light irradiation range, one part of the oblique heliconical cholesteric liquid crystal may be irradiated to transform from the right-handed oblique heliconical cholesteric phase to the left-handed oblique heliconical cholesteric phase, while the other part of the oblique heliconical cholesteric liquid crystal not irradiated remains in the right-handed oblique heliconical cholesteric phase, such that both the left-handed oblique heliconical cholesteric phase and the right-handed oblique heliconical cholesteric phase are simultaneously formed in the oblique heliconical cholesteric liquid crystal, enabling simultaneous reflection of left-handed reflected light and right-handed reflected light, thereby significantly improving the reflection contrast of the oblique heliconical cholesteric liquid crystal.

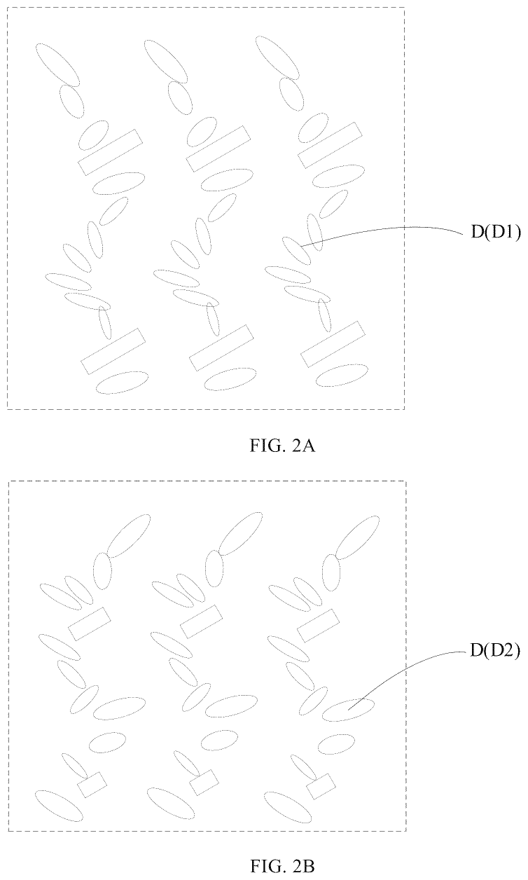

FIG. 2A and FIG. 2B respectively show schematic diagrams of two stable-state structures of oblique heliconical cholesteric liquid crystals according to some embodiments of the present disclosure.

Exemplarily, in some embodiments of the present disclosure, referring to FIG. 2A and FIG. 2B, the oblique heliconical cholesteric liquid crystal D may include a right-handed oblique heliconical cholesteric liquid crystal D1 and a left-handed oblique heliconical cholesteric liquid crystal D2. For example, the initial state of the oblique heliconical cholesteric liquid crystal D added with the chiral photosensitive molecular switch may include the right-handed oblique heliconical cholesteric liquid crystal D1. After light irradiation of a specific wavelength, for example, 365 nm wavelength light or 450 nm wavelength light irradiation, the stable state of the oblique heliconical cholesteric liquid crystal D may transform from the right-handed oblique heliconical cholesteric liquid crystal D1 to the left-handed oblique heliconical cholesteric liquid crystal D2. The helical twisting power (HTP) value of the initial state of the chiral photosensitive molecular switch added in the oblique heliconical cholesteric liquid crystal is small, and when the oblique heliconical cholesteric liquid crystal is irradiated with light of different wavelengths, the helical twisting power of the chiral photosensitive molecule increases sharply and significantly. Due to the excellent helical twisting power change of the chiral photosensitive molecule between the initial state and after light stimulation, a small amount of the chiral photosensitive molecular switch is introduced into the oblique heliconical cholesteric liquid crystal system, and the pitch of the oblique heliconical cholesteric liquid crystal system may be adjusted by the chiral photosensitive molecular switch together with other non-photosensitive chiral dopants, thereby achieving the inversion of left-handedness and right-handedness of the chirality of the oblique heliconical cholesteric liquid crystal system after light irradiation.

The helical twisting power of the chiral photosensitive molecular switch changes differently under light irradiation with different wavelengths. For example, the helical twisting power of the chiral photosensitive molecular switch under light irradiation with wavelength of 450 nm is greater than the helical twisting power of the chiral photosensitive molecular switch under light irradiation with wavelength of 365 nm. The greater change in helical twisting power, the more conducive it is for the chiral inversion of the oblique heliconical cholesteric liquid crystal system.

Exemplarily, in some embodiments of the present disclosure, the chiral photosensitive molecular switch may be an α-cyano-substituted diarylethene molecule, which has a π-conjugated group and good photosensitivity. Z/E isomerization may occur under the stimulation of an external light source. After being connected with a chiral unit, the helical twisting power of the chiral photosensitive molecule may be changed, such that the cholesteric liquid crystal prepared by introducing the α-cyano-substituted diarylethene chiral photosensitive molecular switch may exhibit good light-responsive properties to light sources in specific wavelength bands, ultimately causing the reflection color of the cholesteric liquid crystal to change, while maintaining good thermal stability.

Exemplarily, in some embodiments of the present disclosure, chiral photosensitive molecular switches with different functional groups have different changes in helical twisting power after light irradiation, and thus have different effects on the stable-state transition of the oblique heliconical cholesteric liquid crystal added with the chiral photosensitive molecular switch.

For example, in the chiral photosensitive molecular switch with the chemical structure of general formula I, when other functional group structures are the same, the amount of change in helical twisting power of the chiral photosensitive molecular switch under the same light irradiation conditions when M1 is the structure of

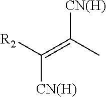

is greater than the amount of change in helical twisting power of the chiral photosensitive molecular switch under the same light irradiation conditions when M1 is the structure of

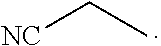

The greater the helical twisting power of the chiral photosensitive molecular switch under light irradiation, the more conducive it is for the oblique heliconical cholesteric liquid crystal added with the chiral photosensitive molecular switch to achieve chiral inversion.

Exemplarily, in some embodiments of the present disclosure, the chemical structure of at least one of the groups R₁ and R₂ in the chiral photosensitive molecular switch is represented by one of the following formulas:

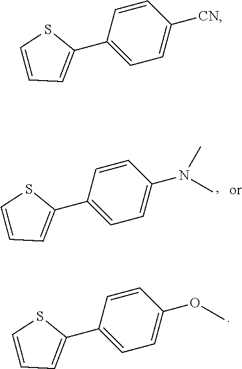

R₁ and R₂ may be the same or different. Groups R₁ and R₂ may be a benzene ring, biphenyl, an aromatic ring or an aromatic heterocyclic ring.

Groups R₁ and R₂ may enable the chiral photosensitive molecular switch to have both chirality and light responsiveness. Introducing groups R₁ and R₂ may increase the conjugation degree of the molecule, thereby endowing the chiral photosensitive molecular switch with controllable light-responsive properties. For example, groups R₁ or R₂ may contain various groups such as —CN, —N, —S, or —O, and these groups will affect the conjugation degree of groups R₁ or R₂. When the conjugation degree increases, the sensitivity redshifts, meaning that the higher the conjugation degree in the chiral photosensitive molecular switch, the longer the wavelength of the irradiation light required to trigger the photosensitive change of the chiral photosensitive molecular switch.

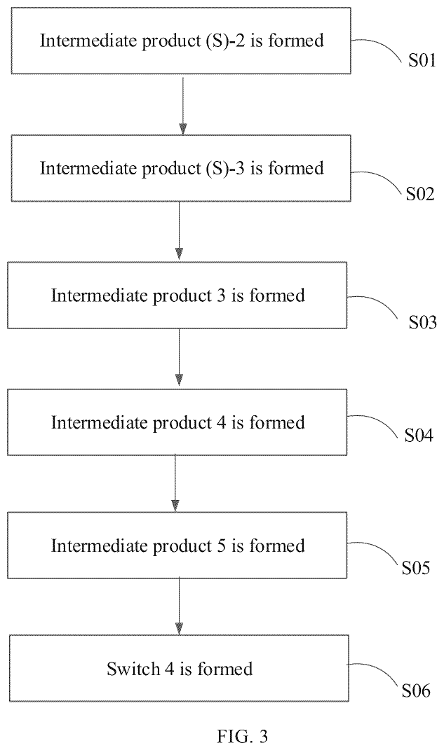

FIG. 3 shows a schematic diagram of a preparation process of a chiral photosensitive molecular switch according to some embodiments of the present disclosure.

Exemplarily, in some embodiments of the present disclosure, the chemical structure of the chiral photosensitive molecular switch may be represented by one of the following formulas:

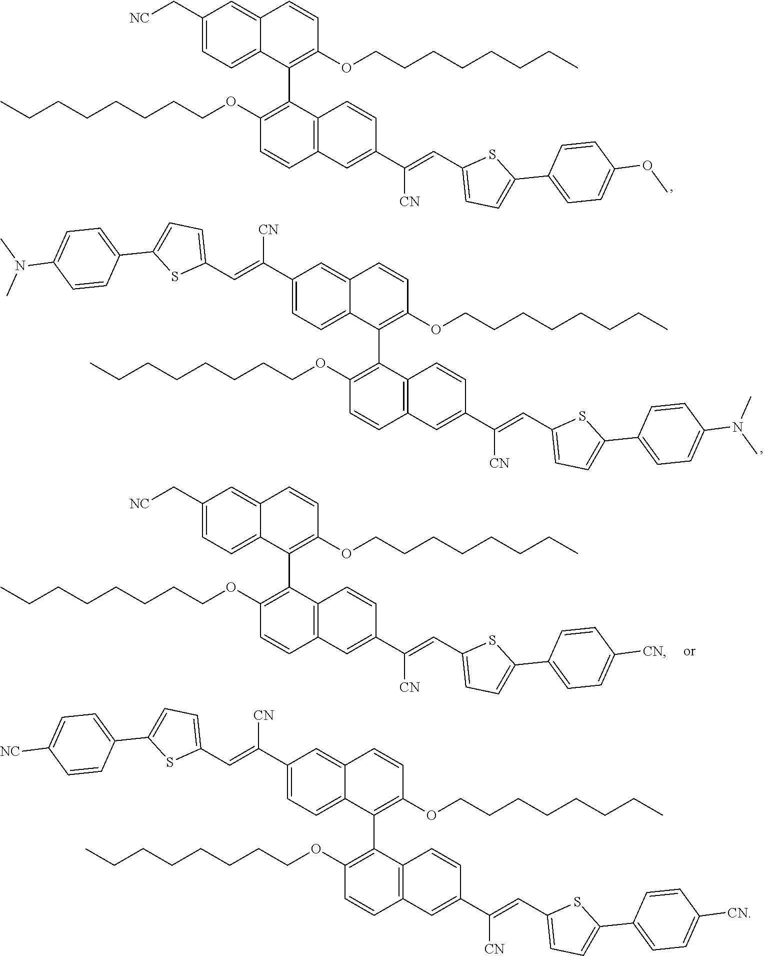

For convenience of description, the chiral photosensitive molecular switches with the above-mentioned four structures are referred to as Switch 1, Switch 2, Switch 3, and Switch 4, respectively.

Exemplarily, in some embodiments of the present disclosure, referring to FIG. 3, the preparation process of Switch 4 may include the following steps S01 to S06.

In Step S01, intermediate product (S)-2 is formed: 6,6′-dibromo-[1,1′-binaphthyl]-2,2′-diol is dissolved in anhydrous acetonitrile solution. After sufficient dissolution, bromooctane and potassium carbonate are weighed and sequentially added to the flask, and reflux is carried out at 80° C. for 24 h. After the reaction is finished, the crude product is preliminarily purified through processes such as extraction, washing, and drying, further purified and separated in a chromatographic column, and dried to obtain the intermediate product (S)-2.

In Step S02, intermediate product (S)-3 is formed: Compound(S)-2 and pre-prepared sodium 2-cyanoacetate are dissolved in 65 ml of anhydrous xylene; allylpalladium chloride dimer and 4,5-bis(diphenylphosphino)-9,9-dimethylxanthene are sequentially added to the above mixture, and the mixture is stirred and refluxed at high temperature under exclusion of oxygen for 8 hours. Afterwards, the mixture is cooled and suction-filtered under reduced pressure, the obtained filtrate is concentrated, and the crude product is purified using column chromatography to finally obtain a yellow oily intermediate product.

In Step S03, intermediate product 3 is formed: 5-bromo-2-thiophenecarboxaldehyde and 4-methoxyphenylboronic acid pinacol ester are weighed and dissolved in an appropriate amount of toluene with stirring; a tetrakis(triphenylphosphine)palladium catalyst, n-propanol, and a 20 wt % sodium carbonate solution are sequentially added, and reflux is carried out at high temperature under nitrogen protection. After the reaction is finished, the crude product is preliminarily purified by methods such as extraction, washing, drying, and concentration, then finely purified using column chromatography, spin-dried, and vacuum-dried at room temperature for 3 hours to obtain a pure white solid.

In Step S04, intermediate product 4 is formed: 5-bromo-2-thiophenecarboxaldehyde and 4-(N,N-dimethylamino)phenylboronic acid pinacol ester are weighed and dissolved in an appropriate amount of toluene with stirring; a tetrakis(triphenylphosphine)palladium catalyst, n-propanol, and a 20 wt % sodium carbonate solution are sequentially added, and reflux is carried out at high temperature under nitrogen protection. After the reaction is finished, the crude product is preliminarily purified by methods such as extraction, washing, drying, and concentration, then finely purified using column chromatography, spin-dried, and vacuum-dried at room temperature for 3 hours to obtain a pure white solid.

In Step S05, intermediate product 5 is formed: 5-bromo-2-thiophenecarboxaldehyde and 4-cyanophenylboronic acid pinacol ester are weighed and dissolved in an appropriate a amount of toluene with stirring; tetrakis(triphenylphosphine)palladium catalyst, n-propanol, and a 20 wt % sodium carbonate solution are sequentially added, and reflux is carried out at high temperature under nitrogen protection. After the reaction is finished, the crude product is preliminarily purified by methods such as extraction, washing, drying, and concentration, then finely purified using column chromatography, spin-dried, and vacuum-dried at room temperature for 3 hours to obtain a pure white solid.

In Step S06, Switch 4 is formed: intermediate product (S)-3 and intermediate product 5 are dissolved in anhydrous THF at a molar ratio of 1:2.5, and then potassium tert-butoxide is slowly added with stirring. The mixture is refluxed at 60° C. Afterwards, a dilute HCl solution is added to the above mixture to quench the reaction and adjust the pH to neutral. The obtained crude product is first subjected to simple preliminary purification, and then finely purified by column chromatography to finally obtain a target product. The entire synthesis process is carried out in the dark. Approximately 2.0 g of solid is finally obtained.

Exemplarily, in some embodiments of the present disclosure, the preparation method of Switch 1 may involve dissolving intermediate product (S)-3 from Step S02 and intermediate product 3 from Step S03 in anhydrous THF at a molar ratio of 1:1, then slowly adding potassium tert-butoxide with stirring. The mixture is refluxed at 60° C. Afterwards, a dilute HCl solution is added to the above mixture to quench the reaction and adjust the pH to neutral. The obtained crude product is first subjected to simple preliminary purification, then finely purified by column chromatography to finally obtain a target product. The entire synthesis process is carried out in the dark.

Exemplarily, in some embodiments of the present disclosure, the preparation method of Switch 2 may involve dissolving intermediate product (S)-3 from Step S02 and intermediate product 4 from Step S04 in anhydrous THF at a molar ratio of 1:2.5, then slowly adding potassium tert-butoxide with stirring. The mixture is refluxed at 60° C. Afterwards, a dilute HCl solution is added to the above mixture to quench the reaction and adjust the pH to neutral. The obtained crude product is first subjected to simple preliminary purification, then finely purified by column chromatography to finally obtain a target product. The entire synthesis process is carried out in the dark.

Exemplarily, in some embodiments of the present disclosure, the preparation method of Switch 3 may involve dissolving intermediate product (S)-3 from Step S02 and intermediate product 5 from Step S05 in anhydrous THF at a molar ratio of 1:1, then slowly adding potassium tert-butoxide with stirring. The mixture is refluxed at 60° C. Afterwards, a dilute HCl solution is added to the above mixture to quench the reaction and adjust the pH to neutral. The obtained crude product is first subjected to simple preliminary purification, then finely purified by column chromatography to finally obtain a target product. The entire synthesis process is carried out in the dark.

Exemplarily, in some embodiments of the present disclosure, the chiral photosensitive molecular switch may be Switch 4, which has a fast light regulation rate and good stability. The oblique heliconical cholesteric liquid crystal added with Switch 4 may quickly achieve chiral inversion under light irradiation, which is further beneficial to the preparation of the oblique heliconical cholesteric liquid crystal display panel.

Exemplarily, at least some embodiments of the present disclosure further provide a cholesteric liquid crystal composition, where the composition may include the above-mentioned chiral photosensitive molecular switch. For example, the composition may include at least one of Switch 1, Switch 2, Switch 3, or Switch 4.

As shown in Table 1 below, the structures of chiral photosensitive molecular switches and ¹H NMR data thereof according to some embodiments of the present disclosure are exemplarily shown.

TABLE 1
Structures of chiral photosensitive molecular switches and ¹H NMR data thereof

| Name | Compound Structure | ¹H NMR data |
|---|---|---|
| Switch 1 | 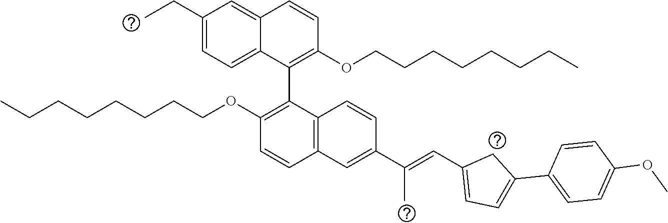 [SMILES: CCCCCCCCOc1ccc2cc(CC)ccc2c1-c1c(OCCCCCCCC)ccc2cc(/C(C)=C/C3=CC=C(c4ccc(OC)cc4)C3)ccc12] | ¹H NMR (C₃D₆O, 400 MHz), δ (ppm): 8.18-8.23 (d, 1H), 7.96-7.90 (t, 1H), 7.83-7.89 (m, 5H), 7.65-7.70 (m, 3H), 7.54-7.59 (s, 1H), 7.15-7.2 (d, 1H), 7.05-7.08 (d, 2H), 6.93-7 (m, 3H), 4.44-4.49 (s, 2H), 4.16-4.2 (t, 4H), 3.81-3.86 (s, 3H), 1.76-1.89 (m, 4H), 1.43-1.48 (m, 4H), 1.3-1.39 (m, 16H), 0.88-0.94 (m, 6H), |
| Switch 2 | 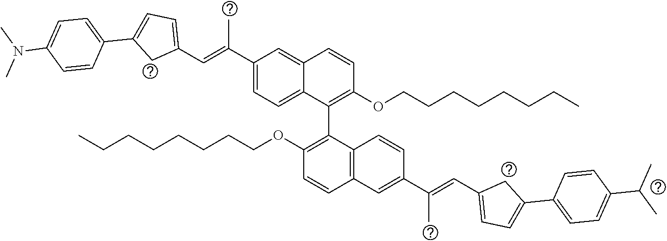 [SMILES: CCCCCCCCOc1ccc2cc(/C(C)=C/C3=CC=C(c4ccc(C(C)C)cc4)C3)ccc2c1-c1c(OCCCCCCCC)ccc2cc(/C(C)=C/C3=CC=C(c4ccc(N(C)C)cc4)C3)ccc12] | ¹H NMR (C₃D₆O, 400 MHz), δ (ppm): 8.18-8.21 (d, 2H) 7.96-7.99 (t, 2H) 7.83-7.89 (m, 4H) 7.65-7.69 (m, 2H) 7.54-7.56 (s, 2H) 7.15-7.2 (d, 2H) 6.85-6.92 (m, 10H) 4.16-4.19 (t, 4H) 3.02-3.08 (s, 12H) 1.76-1.79 (m, 4H) 1.43-1.48 (m, 4H) 1.26-1.3 (m, 16H) 0.84-0.88 (m, 6H) |
| Switch 3 | 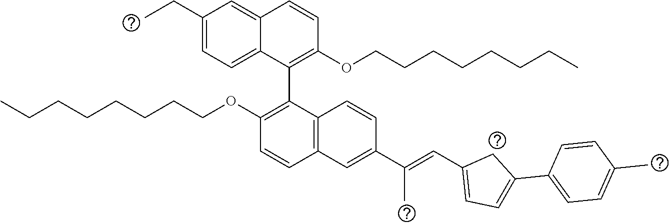 [SMILES: CCCCCCCCOc1ccc2cc(CC)ccc2c1-c1c(OCCCCCCCC)ccc2cc(/C(C)=C/C3=CC=C(c4ccc(C)cc4)C3)ccc12] | ¹H NMR (C₃D₆O, 400 MHz), δ (ppm): 8.15-8.18 (d, 1H), 7.96-7.99 (t, 1H), 7.82-7.86 (m, 9H), 7.65-7.68 (m, 1H), 7.54-7.57 (s, 1H), 7.15-7.18 (d, 1H), 6.9-6.93 (m, 3H), 4.44-4.47 (s, 2H), 4.16-4.19 (t, 4H), 1.76-1.79 (m, 4H), 1.43-1.46 (m, 4H), 1.26-1.3 (m, 16), 0.88-0.85 (m, 6H), |
| Switch 4 | 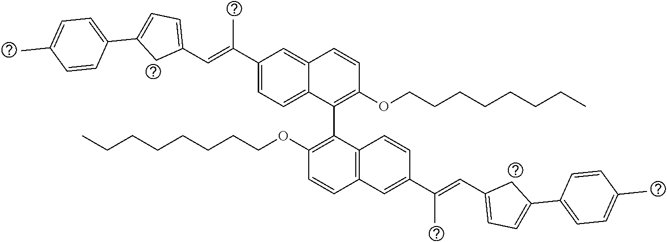 [symbol] | ¹H NMR (C₃D₆O, 400 MHz), δ (ppm): 8.15-8.18 (d, 2H), 7.96-7.98 (t, 2H), 7.77-7.84 (m, 12H), 7.65-7.68 (m, 2H), 7.54-7.58 (s, 2H), 7.15-7.19 (d, 2H), 6.93-6.95 (d, 2H), 4.16-4.19 (t, 4H), 1.76-1.78 (m, 4H), 1.43-1.47 (m, 4H), 1.3-1.33 (m, 16H), 0.88-0.85 (m, 6H), |

[symbol]

indicates data missing or illegible when filed

Exemplarily, in some embodiments of the present disclosure, the composition may further include a bent-core molecular mixture, a rod-like single crystal mixture, and a non-photosensitive chiral dopant.

In the presence of an organic solvent, the bent-core molecular mixture, the rod-like single crystal molecular mixture, the non-photosensitive chiral dopant, and the chiral photosensitive molecular switch are mixed uniformly, and the solvent is evaporated to obtain the cholesteric liquid crystal composition. The organic solvent may include at least one of acetone, methanol, ethanol, tetrahydrofuran, dichloromethane, or chloroform.

Exemplarily, in some embodiments of the present disclosure, a proportion of the chiral photosensitive molecular switch is 0.5% to 10% by the weight of the cholesteric liquid crystal composition.

Exemplarily, in some embodiments of the present disclosure, the chiral photosensitive molecular switch may include at least one of Switch 1, Switch 2, Switch 3, or Switch 4.

Exemplarily, in some embodiments of the present disclosure, a proportion of the non-photosensitive chiral dopant is 0.5% to 10% by the weight of the cholesteric liquid crystal composition. For example, the non-photosensitive chiral dopant may include at least one of S811, R811, S5011, or R5011.

The molecular structural formula of S811 is as follows:

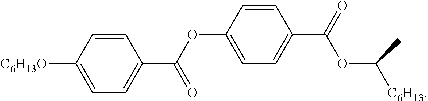

The molecular structural formula of R811 is as follows:

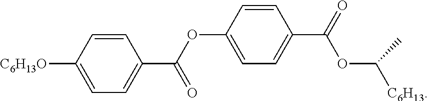

The molecular structural formula of S5011 is as follows:

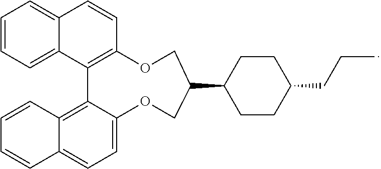 [SMILES: CCC[C@H]1CC[C@H](C2COc3ccc4ccccc4c3-c3c(ccc4ccccc34)OC2)CC1]

The molecular structural formula of R5011 is as follows:

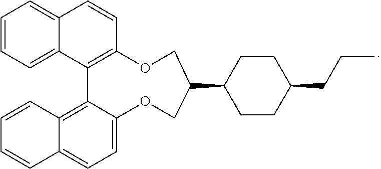 [SMILES: CCC[C@H]1CC[C@@H](C2COc3ccc4ccccc4c3-c3c(ccc4ccccc34)OC2)CC1]

Exemplarily, in some embodiments of the present disclosure, a proportion of the bent-core molecular mixture is 30% to 70% by the weight of the cholesteric liquid crystal composition, and/or a proportion of the rod-like single crystal mixture is 30% to 70% by the weight of the cholesteric liquid crystal composition.

The bent-core molecular mixture may be a mixture of Compound 1, Compound 2, and Compound 3.

The molecular structural formula of Compound 1 is as follows:

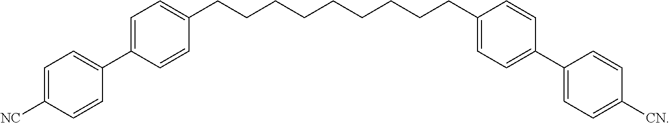 [SMILES: N#Cc1ccc(-c2ccc(CCCCCCCCCc3ccc(-c4ccc(C#N)cc4)cc3)cc2)cc1]

The molecular structural formula of Compound 2 is as follows:

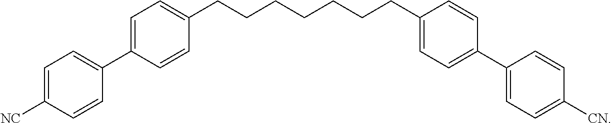 [SMILES: N#Cc1ccc(-c2ccc(CCCCCCCc3ccc(-c4ccc(C#N)cc4)cc3)cc2)cc1]

The molecular structural formula of Compound 3 is as follows:

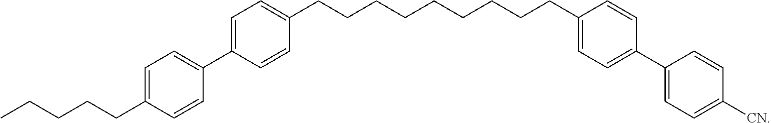 [SMILES: CCCCCc1ccc(-c2ccc(CCCCCCCCCc3ccc(-c4ccc(C#N)cc4)cc3)cc2)cc1]

For example, by the total weight of the bent-core molecular mixture, the content of Compound 1 is 77%, the content of Compound 2 is 13%, and the content of Compound 3 is 10%. Mixing Compound 1, Compound 2, and Compound 3 in proportion may obtain a bent-core molecular mixture capable of inducing an oblique heliconical cholesteric liquid crystal material.

As shown in Table 2 below, the structures of compounds and ¹H NMR data thereof according to some embodiments of the present disclosure are exemplarily shown.

TABLE 2
Compound Structures and ¹H NMR Data thereof

| Name | Compound Structure | ¹H NMR Data |
|---|---|---|
| Compound 1 | 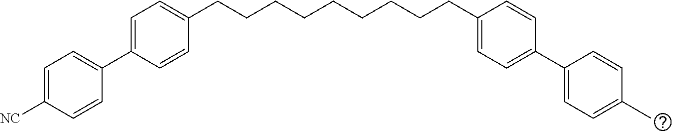 [SMILES: Cc1ccc(-c2ccc(CCCCCCCCCc3ccc(-c4ccc(C#N)cc4)cc3)cc2)cc1] | ¹H NMR (C₃D₆O, 400 MHz), δ (ppm): 7.84- 7.86 (s, 8H), 7.60-7.62 (d, 4H), 7.28-7.31 (d, 4H), 2.63-2.67 (t, 4H), 1.63-1.66 (m, 4H), 1.26-1.24 (t, 10H) |
| Compound 2 | 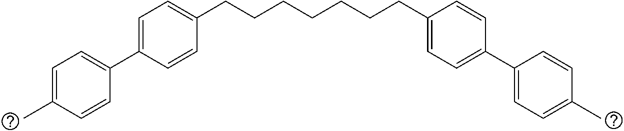 | ¹H NMR (C₃D₆O, 400 MHz), δ (ppm): 7.80-7.86 (m, 8H), 7.62-7.66 (d, 4H), 7.32-7.36 (d, 4H), 2.63-2.68 (t, 4H), 1.61-1.67 (m, 4H), 1.35-1.40 (m, 6H) |
| Compound 3 | 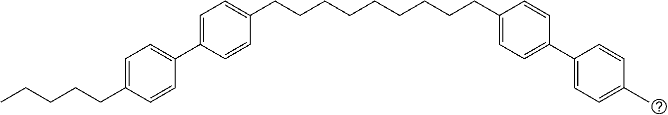 [SMILES: CCCCCc1ccc(-c2ccc(CCCCCCCCCc3ccc(-c4ccc(C)cc4)cc3)cc2)cc1] [symbol] | ¹H NMR (C₃D₆O, 400 MHz), δ (ppm): 7.81-7.85 (s, 4H), 7.61-7.65 (d, 6H), 7.26-7.32 (d, 6H), 2.62-2.66 (m, 6H), 1.59-1.64 (m, 6H), 1.24-1.32 (m, 14H), 0.90-0.93 (m, 3H) |

[symbol]

indicates data missing or illegible when filed

Exemplarily, the rod-like single crystal mixture may be a mixture of a single crystal molecule 1 and a single crystal molecule 2.

The molecular structural formula of the single crystal molecule 1 is as follows:

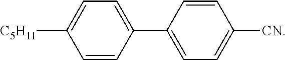

The molecular structural formula of the single crystal molecule 2 is as follows:

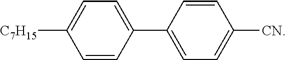

For example, by the total weight of the single crystal molecular mixture, the content of the single crystal molecule 1 is 84%, and the content of the single crystal molecule 2 is 16%. Mixing the single crystal molecule 1 and the single crystal molecule 2 in proportion may obtain the single crystal molecular mixture.

In the presence of an organic solvent, the bent-core molecular mixture, the rod-like single crystal molecular mixture, the non-photosensitive chiral dopant, and the chiral photosensitive molecular switch described above are mixed uniformly in a certain proportion, and the solvent is evaporated to obtain a cholesteric liquid crystal composition capable of inducing oblique helices upon energization.

In some preferred embodiments, by the weight of the cholesteric liquid crystal composition, a proportion of the bent-core molecular mixture is 30% to 70%, a proportion of the rod-like single crystal mixture is 30% to 70%, a proportion of the chiral photosensitive molecular switch is 5% to 10%, a proportion of the non-photosensitive chiral dopant is 5% to 10%, and the sum of the proportion of the bent-core molecular mixture, the proportion of the rod-like single crystal mixture, the proportion of the chiral photosensitive molecular switch and the proportion of the non-photosensitive chiral dopant is 100%.

For example, the bent-core molecular mixture, the rod-like single crystal molecular mixture, the non-photosensitive chiral dopant, and the chiral photosensitive molecular switch described above are respectively mixed in a weight ratio of 50:40:5:5, and after evaporating the solvent, a cholesteric liquid crystal composition capable of inducing oblique helices upon energization is obtained.

Table 3 shows the experimental performance of cholesteric liquid crystal compositions under different proportions. In Table 3, “Liquid Crystal 1” and “Liquid Crystal 2” respectively represent rod-like single crystal molecular mixtures with different structures or proportions, and they, for example, may be mixtures of the above-mentioned single crystal molecule 1 and single crystal molecule 2 in different proportions. The numerical values under the columns of “Liquid Crystal 1”, “Liquid Crystal 2”, “Bent-core Molecular Mixture”, “Chiral Dopant”, and “Switch 4” respectively represent the weight percentages of the corresponding substances in the cholesteric liquid crystal composition. It may be seen from the experimental performance in Table 3 that the experimental performance of Group 3 is better, i.e., by the weight of the cholesteric liquid crystal composition, the proportion of the bent-core molecular mixture is 50%, the proportion of the rod-like single crystal mixture is 40%, the proportion of the chiral photosensitive molecular switch is 5%, and the proportion of the non-photosensitive chiral dopant is 5%, and the following experimental phenomena are observed: data and inversion may be measured; after a plurality of repetitions, all exhibit excellent reflection performance.

TABLE 3
Experimental Performance of Cholesteric Liquid Crystal Compositions under Different Proportions

| | | | Bent-core | | | |
|---|---|---|---|---|---|---|
| | Liquid | Liquid | Molecular | Chiral | | |
| Group | Crystal 1 | Crystal 2 | Mixture | Dopant | Switch 4 | Phenomenon |
| 1 | 40 | / | 50 | 2 | 2 | No reflection color |
| 2 | 40 | / | 50 | 3 | 3 | No reflection color |
| 3 | 40 | / | 50 | 5 | 5 | Data and inversion may be measured; |
| | | | | | | after a plurality of repetitions, all |
| | | | | | | exhibit excellent reflection |
| | | | | | | performance |
| 4 | / | 40 | 50 | 5 | 3.49 | No reflection color |
| 5 | / | 40 | 50 | 5 | 4.49 | No reflection color |
| 6 | / | 40 | 50 | 2 | 1.93 | There is reflection color at first; |
| | | | | | | attempts to change irradiation time |
| | | | | | | many times fail to achieve inversion |

Exemplarily, the cholesteric liquid crystal composition capable of inducing oblique helices upon energization may regulate the handedness of oblique heliconical cholesteric liquid crystals with light and regulate the reflection band with an electric field, and may be used to prepare oblique heliconical cholesteric liquid crystals with high reflectivity, high contrast, and good stability, which may be applied to electric field-regulated reflection in the full spectral range.

Furthermore, the cholesteric liquid crystal composition capable of inducing oblique helices upon energization may be used to prepare the cholesteric liquid crystal display panel with high reflectivity and high contrast. Within a certain range, the higher the content of chiral agents in the cholesteric liquid crystal composition, the higher the reflection contrast of the cholesteric liquid crystal display panel prepared with the cholesteric liquid crystal composition, and the better the display effect.

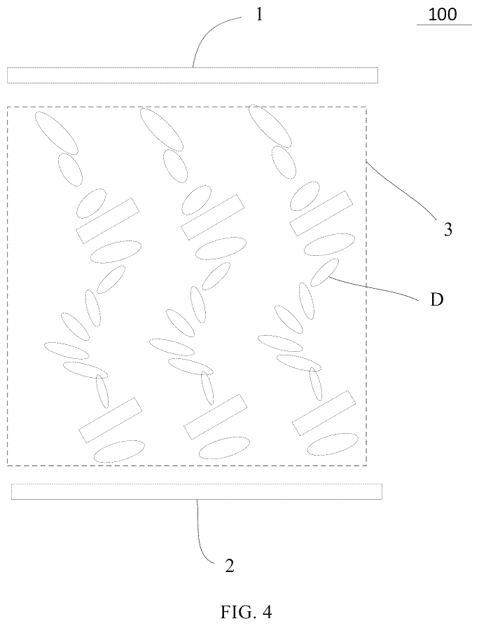

FIG. 4 shows a schematic structural diagram of a cholesteric liquid crystal display panel according to some embodiments of the present disclosure.

Referring to FIG. 4, at least some embodiments of the present disclosure further provide a cholesteric liquid crystal display panel, where the display panel 100 includes: a first substrate 1 and a second substrate 2 arranged opposite to each other; a cholesteric liquid crystal layer 3 located between the first substrate 1 and the second substrate 2, where the cholesteric liquid crystal layer 3 includes a cholesteric liquid crystal composition, and the cholesteric liquid crystal composition includes the above-mentioned chiral photosensitive molecular switch, or the cholesteric liquid crystal composition is the above-mentioned composition. Due to the presence of the chiral photosensitive molecular switch in the cholesteric liquid crystal composition, photoisomerization may occur under the regulation of light irradiation to achieve chiral inversion.

Exemplarily, continuing to refer to FIG. 4, the cholesteric liquid crystal layer 3 may include the oblique heliconical cholesteric liquid crystal D, where the oblique heliconical cholesteric liquid crystal D may be obtained by pouring the cholesteric liquid crystal composition into a conductive liquid crystal cell at a certain temperature, cooling it to room temperature at a certain rate, applying an external voltage to convert the cholesteric liquid crystal in the liquid crystal cell into a field-induced nematic state, and then decreasing the voltage, so that the cholesteric liquid crystal with the oblique heliconical arrangement may be obtained. For example, the cooling rate may be 0.5-3° C./min, and the external voltage may be 75-100 V.

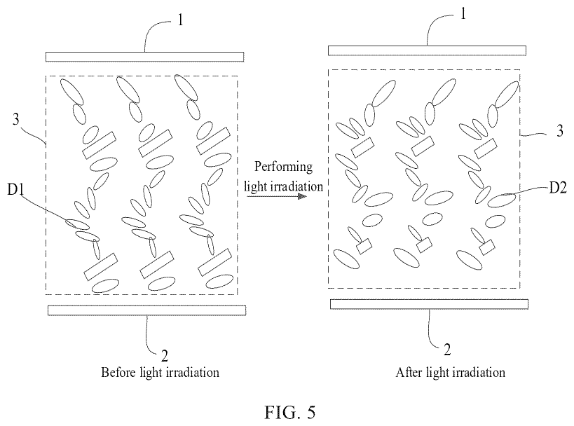

FIG. 5 shows a schematic diagram of a comparison between structures of a cholesteric liquid crystal display panel before and after light irradiation according to some embodiments of the present disclosure.

Exemplarily, in some embodiments of the present disclosure, referring to FIG. 5, before light irradiation, the cholesteric liquid crystal layer 3 of the cholesteric liquid crystal display panel may include the right-handed oblique heliconical cholesteric liquid crystal D1. When the cholesteric liquid crystal layer 3 is irradiated with light of a specific wavelength band, photoisomerization occurs due to the presence of the chiral photosensitive molecular switch in the cholesteric liquid crystal layer 3, causing chiral inversion of the cholesteric liquid crystal layer 3, where the right-handed oblique heliconical cholesteric liquid crystal D1 is converted into left-handed oblique heliconical cholesteric liquid crystal D2.

The chiral inversion of the oblique heliconical cholesteric liquid crystal D may be achieved by light irradiation, and the transformation from right-handed oblique heliconical cholesteric liquid crystal D1 to left-handed oblique heliconical cholesteric liquid crystal D2 is achieved, and may stably exist. Further, by designing the light irradiation range, one part of the oblique heliconical cholesteric liquid crystal may be irradiated to transform from the right-handed oblique heliconical cholesteric phase to the left-handed oblique heliconical cholesteric phase, while the other part of the oblique heliconical cholesteric liquid crystal not irradiated remains in the right-handed oblique heliconical cholesteric phase, such that both left-handed and right-handed oblique heliconical cholesteric liquid crystals are simultaneously formed in the oblique heliconical cholesteric liquid crystal D, enabling simultaneous reflection of left-handed and right-handed reflected light, thereby significantly improving the reflection contrast of the oblique heliconical cholesteric liquid crystal.

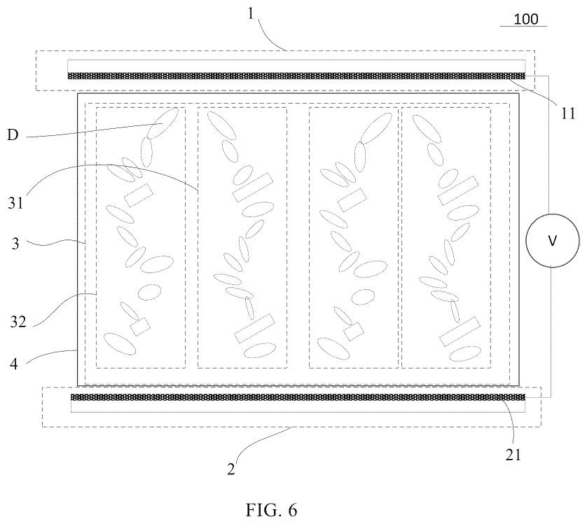

FIG. 6 shows a schematic structural diagram of a cholesteric liquid crystal display panel according to some embodiments of the present disclosure.

Exemplarily, in some embodiments of the present disclosure, referring to FIG. 6, the cholesteric liquid crystal layer 3 includes a first cholesteric liquid crystal unit 31 and a second cholesteric liquid crystal unit 32. The liquid crystal in the first cholesteric liquid crystal unit 31 has an oblique heliconical structure with a first handedness, and the liquid crystal in the second cholesteric liquid crystal unit 32 has an oblique heliconical structure with a second handedness, where the first handedness is opposite to the second handedness. For example, the first handedness may be left-handed, and the corresponding second handedness may be right-handed; alternatively, the first handedness may be right-handed, and the corresponding second handedness may be left-handed. For example, the cholesteric liquid crystal display panel may simultaneously include both the left-handed oblique heliconical cholesteric liquid crystal and the right-handed oblique heliconical cholesteric liquid crystal.

Since the left-handed and right-handed oblique heliconical cholesteric liquid crystals are simultaneously formed in the cholesteric liquid crystal display panel, both the left-handed reflected light and the right-handed reflected light may be simultaneously reflected, thereby significantly improving the reflection contrast of the oblique heliconical cholesteric liquid crystal display panel.

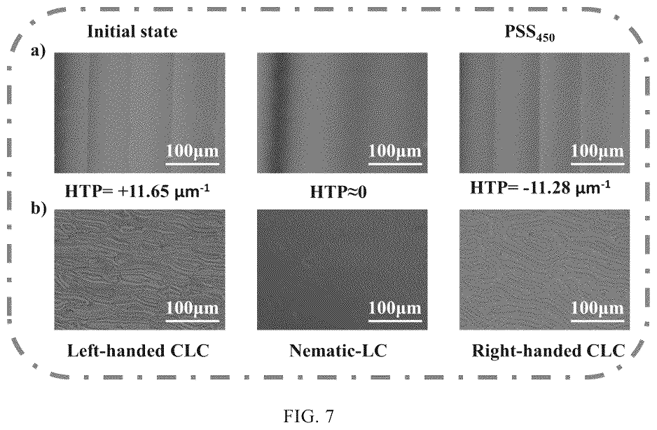

FIG. 7 shows a schematic diagram of a comparison between helical twisting powers of an oblique heliconical cholesteric liquid crystal system added with a chiral photosensitive molecular switch in different states according to some embodiments of the present disclosure.

Referring to FIG. 7, FIG. 7a) and FIG. 7b) respectively show Cano line changes and phase state changes of the cholesteric liquid crystal system doped with Switch 4 in different states, observed by a polarizing microscope.

It should be noted that in the art, the Grandjean-Cano method may be used to determine the chiral size of a molecule, i.e., the HTP value. For example, an appropriate amount of Switch 4 may be weighed and added to the liquid crystal host to prepare a liquid crystal mixture system with a concentration of 5 wt %, then dichloromethane is added, and ultrasonic waves are applied for 10 min so that it may fully dissolved, and then the liquid crystal mixed system is placed in a vacuum oven for drying. After drying, it is poured into a wedge-shaped liquid crystal cell at room temperature, and the Cano line changes of each photostationary state are observed under a polarizing microscope, and the Cano line spacing is recorded.

In embodiments of the present disclosure, the HTP values of the liquid crystal system in various states may be calculated to quantitatively analyze the light-regulated performance of chirality.

As shown in the leftmost a) of FIG. 7, in the initial state, the HTP value may be calculated to be +11.65 from the Cano line spacing. The liquid crystal system is poured into a vertically oriented liquid crystal cell with a thickness of 10 μm and observed under a polarizing microscope, as shown in leftmost b) of FIG. 7, where a typical cholesteric fingerprint texture may be clearly seen. According to the HTP value and the CD test result, it may be concluded that Switch 4 induces a left-handed helical structure at this point.

Switch 4 molecules are very sensitive to 365 nm and 450 nm light sources. In the initial state, irradiating the Switch 4 molecules with a 365 nm or 450 nm light source for 30 seconds will cause the molecules to isomerize to the PSS₃₆₅ or PSS₄₅₀ state.

The middle and rightmost a) and b) in FIG. 7 show Cano line changes and phase state changes during the transformation of the liquid crystal system from the initial state to the PSS₄₅₀ state. For PSS₄₅₀, the Cano lines gradually disappear from the initial state, referring to the middle a) in FIG. 7, HTP=0; then the Cano lines slowly reappear, forming new Cano lines with a wider spacing, referring to the rightmost a) of FIG. 7, HTP=−11.28; the same phenomenon occurs in the phase state: the fingerprint texture first gradually disappears, referring to the middle b) of FIG. 7, then a new, sparser fingerprint texture is formed, referring to the rightmost b) of FIG. 7. These phenomena fully indicate that the liquid crystal system undergoes phase transition and chiral inversion under blue light induction, and this is because the chirality of the acceptor in PSS₄₅₀ increases sharply and exceeds the sum of the chirality of the two donors, and thus the chirality of the assembly is dominated by the acceptor part, thereby inducing a right-handed cholesteric helical structure.

It may be seen from the experimental data in FIG. 7 that Switch 4 has a fast light regulation rate and good stability. Moreover, the molecular chirality changes and may stably exist after light irradiation. Co-doping the system with Switch 4 and a chiral agent of the opposite handedness easily achieve chiral inversion between the initial state and PSS₄₅₀, with the absolute values of the HTP values of the system before and after chiral inversion being similar.

Exemplarily, in some embodiments of the present disclosure, the absolute value of the helical twisting power of the liquid crystal in the first cholesteric liquid crystal unit is substantially equal to the absolute value of the helical twisting power of the liquid crystal in the second cholesteric liquid crystal unit.

It should be noted that in the text, the expression “the absolute values of the helical twisting powers (i.e., HTP) are simultaneously equal or similar” includes at least the following situations: the absolute values of the two helical twisting powers (i.e., HTP) being compared are strictly equal; or the ratio of the absolute values of the two helical twisting powers (i.e., HTP) being compared is between 0.8 and 1.2.

For example, in some embodiments of the present disclosure, the chiral photosensitive molecular switch may be Switch 4, which is very sensitive to 365 nm and 450 nm light sources. In the initial state, irradiating the oblique heliconical cholesteric liquid crystal doped with the chiral photosensitive molecular switch with a 365 nm or 450 nm light source for 30 seconds will cause the liquid crystal to isomerize to the PSS₃₆₅ or PSS₄₅₀ state, where PSS₃₆₅ refers to the stable state of the liquid crystal after chiral inversion under 365 nm wavelength light irradiation, and PSS₄₅₀ refers to the stable state of the liquid crystal after chiral inversion under 450 nm wavelength light irradiation.

After irradiation with a specific wavelength, the chirality of the oblique heliconical cholesteric liquid crystal doped with the chiral photosensitive molecular switch changes and may stably exist after irradiation. For example, referring to FIG. 7, co-doping the oblique heliconical cholesteric liquid crystal system with Switch 4 and a chiral agent of the opposite handedness may easily achieve chiral inversion of the liquid crystal between the initial state and the PSS₄₅₀ state, with the absolute values of the HTP values of the system before and after chiral inversion being similar, for example, the absolute value of HTP in the initial state is 11.65 μm⁻¹, and the absolute value of HTP in the PSS₄₅₀ state is 11.28 μm⁻¹. The similar absolute values of HTP in the two stable states before and after inversion are conducive to further designing the oblique heliconical cholesteric liquid crystal display panel with high reflectivity.

Exemplarily, in some embodiments of the present disclosure, continuing to refer to FIG. 6, the display panel further includes: a first electrode 11 located on the first substrate 1 and a second electrode 21 located on the second substrate 2; the cholesteric liquid crystal layer 3 is configured such that the wavelength corresponding to the reflection peak of the cholesteric liquid crystal layer 3 changes in response to a change in a voltage V applied between the first electrode 11 and the second electrode 21. For example, the first electrode 11 and the second electrode 21 may be ITO transparent conductive electrodes, that is, the first substrate 1 and the second substrate 2 may be single-sided conductive substrates.

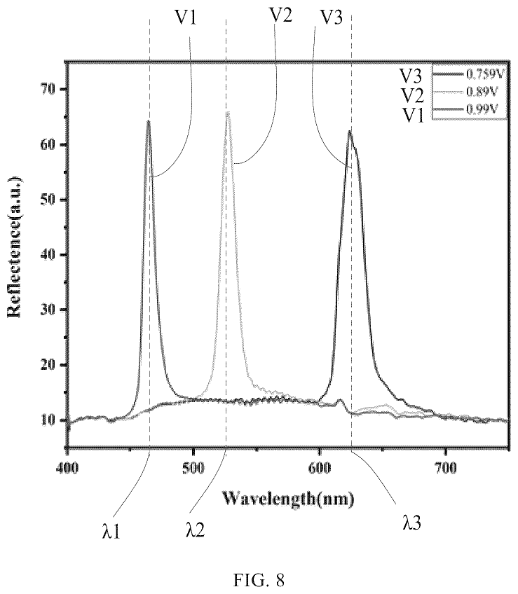

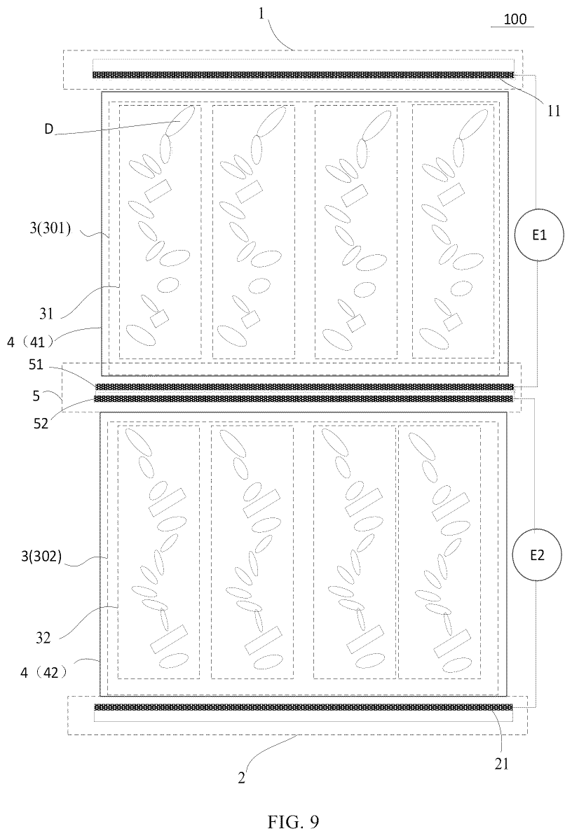

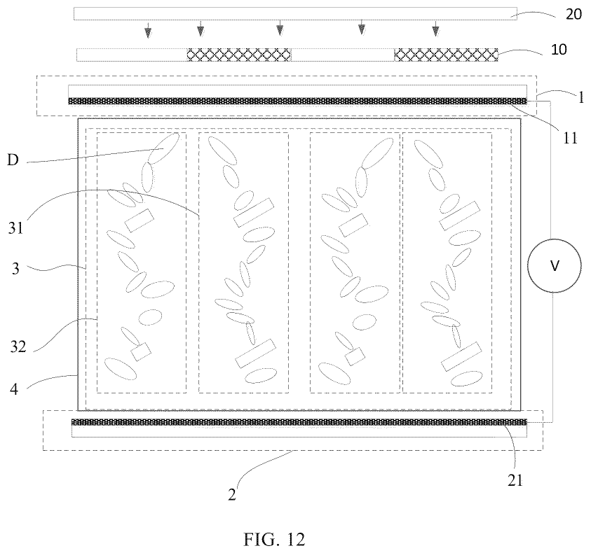

In some exemplary embodiments of the present disclosure, in dichloromethane, the bent molecules capable of inducing the oblique heliconical cholesteric liquid crystal material represented by Compound 1, Compound 2, and Compound 3 in Table 2 are mixed to obtain the bent-core molecular mixture, for example, by the total weight of the bent-core molecular mixture, the content of Compound 1 is 77%, the content of Compound 2 is 13%, and the content of Compound 3 is 10%. The above-mentioned single crystal molecule 1 and the above-mentioned single crystal molecule 2 are mixed to obtain the single crystal molecular mixture, for example, by the total weight of the single crystal molecular mixture, the content of the single crystal molecule 1 is 84%, and the content of the single crystal molecule 2 is 16%. The bent-core molecular mixture, the single crystal molecular mixture, the non-photosensitive chiral dopant, and the chiral photosensitive molecular switch Switch 4 described above are mixed in a weight ratio of 40:50:5:5, and after evaporating the solvent, a cholesteric liquid crystal mixture capable of inducing oblique helices upon energization is obtained. Two samples are respectively poured at a higher temperature into a stacked liquid crystal cell formed by oppositely bonding two single-sided conductive ITO-coated glass substrates subjected to planar alignment treatment and one double-sided conductive ITO-coated glass substrate subjected to planar alignment treatment together (as shown in FIG. 9), or into a liquid crystal cell formed by two single-sided conductive ITO-coated glass substrates subjected to planar alignment treatment (as shown in FIG. 6), and cooled to room temperature at a cooling rate of 1-5° C./min for testing. The prepared liquid crystal cell is layered and irradiated with 450 nm blue light (as shown in FIG. 5) or irradiated under a mask (as shown in FIG. 12). The chiral optical switch molecules exposed to light irradiation undergo configuration changes, thereby causing the liquid crystal system exposed to light irradiation to achieve structural changes in chiral handedness. An external electric field is used to induce the oblique heliconical arrangement of cholesteric liquid crystal molecules (as shown in FIG. 6 and FIG. 9). First, the reflection colors in a case that left-handed and right-handed helices exist alone are observed. Then, the reflection colors in a case that both left-handed and right-handed helices exist simultaneously in the system are observed (as shown in FIG. 8).

FIG. 8 shows a schematic diagram of a relationship between a reflection peak of a cholesteric liquid crystal display panel and an applied voltage according to some embodiments of the present disclosure. It should be noted that in FIG. 8, the abscissa Wavelength (nm) represents wavelength (nm), and the ordinate Reflectance (a.u.) represents reflected light intensity.

Exemplarily, in some embodiments of the present disclosure, referring to FIG. 6 and FIG. 8, the cholesteric liquid crystal layer 3 is configured such that:

in response to a first voltage V1 applied between the first electrode 11 and the second electrode 21, the reflection peak of the cholesteric liquid crystal layer 3 corresponds to a first wavelength 21;

in response to a second voltage V2 applied between the first electrode 11 and the second electrode 21, the reflection peak of the cholesteric liquid crystal layer 3 corresponds to a second wavelength 22; and in response to a third voltage V3 applied between the first electrode 11 and the second electrode 21, the reflection peak of the cholesteric liquid crystal layer 3 corresponds to a third wavelength 23, where the first voltage V1, the second voltage V2, and the third voltage V3 are different from each other, and the first wavelength 21, the second wavelength 22, and the third wavelength 23 correspond to visible light of different colors respectively.

Exemplarily, in some embodiments of the present disclosure, the cholesteric liquid crystal layer 3 is configured such that the wavelength corresponding to the reflection peak of the cholesteric liquid crystal layer increases in response to a decrease in the voltage V applied between the first electrode 11 and the second electrode 21.

Exemplarily, continuing to refer to FIG. 6 and FIG. 8, the first voltage V1 may be greater than the second voltage V2; correspondingly, the first wavelength 21 corresponding to the reflection peak of the cholesteric liquid crystal layer 3 in response to the first voltage V1 applied between the first electrode 11 and the second electrode 21 may be less than the second wavelength 22 corresponding to the reflection peak of the cholesteric liquid crystal layer 3 in response to the second voltage V2 applied between the first electrode 11 and the second electrode 21. The second voltage V2 may be greater than the third voltage V3; correspondingly, the second wavelength 22 corresponding to the reflection peak of the cholesteric liquid crystal layer 3 in response to the second voltage V2 applied between the first electrode 11 and the second electrode 21 may be less than the third wavelength 23 corresponding to the reflection peak of the cholesteric liquid crystal layer 3 in response to the third voltage V3 applied between the first electrode 11 and the second electrode 21.

Exemplarily, the first voltage V1 may be 0.99 V, the second voltage V2 may be 0.89 V, the third voltage V3 may be 0.759 V, the visible light corresponding to the first wavelength 21 may be blue light, the visible light corresponding to the second wavelength 22 may be green light, and the visible light corresponding to the third wavelength 23 may be red light.

Exemplarily, in some embodiments of the present disclosure, continuing to refer to FIG. 6, the first substrate 1 and the second substrate 2 are cell-assembled to form an accommodating space 4, the cholesteric liquid crystal layer 3 is located in the accommodating space 4, and orthographic projections of the first cholesteric liquid crystal unit 31 and the second cholesteric liquid crystal unit 32 on the first substrate 1 are distributed alternately. The liquid crystal in the first cholesteric liquid crystal unit 31 has an oblique heliconical structure with a first handedness, and the liquid crystal in the second cholesteric liquid crystal unit 32 has an oblique heliconical structure with a second handedness, where the first handedness is opposite to the second handedness. For example, the first handedness may be left-handed, and the corresponding second handedness may be right-handed; alternatively, the first handedness may be right-handed, and the corresponding second handedness may be left-handed. That is, the cholesteric liquid crystal display panel may simultaneously include both the left-handed oblique heliconical cholesteric liquid crystal and the right-handed oblique heliconical cholesteric liquid crystal.

By simultaneously forming the left-handed and right-handed oblique heliconical cholesteric phases in the cholesteric liquid crystal display panel, the left-handed reflected light and the right-handed reflected light may be simultaneously reflected, thereby significantly improving the reflection contrast of the oblique heliconical cholesteric liquid crystal.

The alternating distribution of the orthographic projections of the first cholesteric liquid crystal unit 31 and the second cholesteric liquid crystal unit 32 on the first substrate 1 may ensure that the intensity of left-handed reflected light is substantially equal to the intensity of right-handed reflected light, thereby ensuring that the brightness of the cholesteric liquid crystal display panel observed at different positions is substantially the same, which may improve the display effect of the display panel.

FIG. 9 shows a schematic diagram of a stacked structure of a cholesteric liquid crystal display panel according to some embodiments of the present disclosure.

Exemplarily, in some embodiments of the present disclosure, referring to FIG. 9, the display panel 100 may further include a third substrate 5 located between the first substrate 1 and the second substrate 2. The cholesteric liquid crystal layer 3 includes: a first sub-cholesteric liquid crystal layer 301 located between the first substrate 1 and the third substrate 5; and a second sub-cholesteric liquid crystal layer 302 located between the third substrate 5 and the second substrate 2. The first sub-cholesteric liquid crystal layer 301 includes the first cholesteric liquid crystal unit 31, and the second sub-cholesteric liquid crystal layer 302 includes the second cholesteric liquid crystal unit 32. The liquid crystal in the first cholesteric liquid crystal unit 31 may have an oblique heliconical structure with a first handedness, and the liquid crystal in the second cholesteric liquid crystal unit 32 may have an oblique heliconical structure with a second handedness, where the first handedness is opposite to the second handedness. For example, the first handedness may be left-handed, and the corresponding second handedness may be right-handed; alternatively, the first handedness may be right-handed, and the corresponding second handedness may be left-handed. For example, the cholesteric liquid crystal display panel may include both the left-handed oblique heliconical cholesteric liquid crystal and the right-handed oblique heliconical cholesteric liquid crystal. The third substrate 5 may include a third electrode 51 and a fourth electrode 52 located on the upper and lower surfaces of the third substrate 5. For example, the third electrode 51 and the fourth electrode 52 may be ITO transparent conductive electrodes, that is, the third substrate 5 may be a double-sided conductive substrate. By adjusting a voltage E1 between the first electrode 11 and the third electrode 51, the state of the cholesteric liquid crystal in the first sub-cholesteric liquid crystal layer 301 located between the first substrate 1 and the third substrate 5 may be adjusted; by adjusting a voltage E2 between the second electrode 21 and the fourth electrode 52, the state of the cholesteric liquid crystal in the second sub-cholesteric liquid crystal layer 302 located between the third substrate 5 and the second substrate 2 may be adjusted, thereby achieving the electric field regulation of different liquid crystal layers in the stacked liquid crystal display panel.

Exemplarily, by designing the light irradiation range, the first sub-cholesteric liquid crystal layer 301 located between the first substrate 1 and the third substrate 5 may be irradiated to transform from a right-handed cholesteric phase to a left-handed cholesteric phase, while the part of the first sub-cholesteric liquid crystal layer 301 located between the second substrate 2 and the third substrate 5 that is not irradiated remains in the right-handed cholesteric phase, such that both the left-handed cholesteric phase and the right-handed cholesteric phase are simultaneously formed in the oblique heliconical cholesteric liquid crystal, enabling simultaneous reflection of left-handed reflected light and right-handed reflected light, thereby significantly improving the reflection contrast of the oblique heliconical cholesteric liquid crystal.

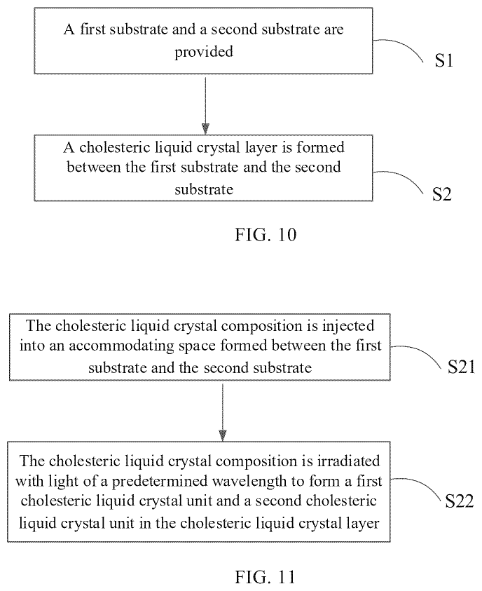

FIG. 10 shows a flowchart of preparing a cholesteric liquid crystal display panel according to some embodiments of the present disclosure.

Referring to FIG. 10, at least some embodiments of the present disclosure further provide a method of preparing a cholesteric liquid crystal display panel, where the method includes the following steps S1-S2.

In Step S1, a first substrate and a second substrate are provided.

In Step S2, a cholesteric liquid crystal layer is formed between the first substrate and the second substrate.

The cholesteric liquid crystal layer includes a cholesteric liquid crystal composition, and the cholesteric liquid crystal composition includes the above-mentioned chiral photosensitive molecular switch, or the cholesteric liquid crystal composition is the above-mentioned composition.

FIG. 11 shows a flowchart of forming a cholesteric liquid crystal layer between a first substrate and a second substrate in step S2 of FIG. 10.

Exemplarily, forming a cholesteric liquid crystal layer between the first substrate and the second substrate in Step S2 specifically includes the following steps S21-S22.

In Step S21, the cholesteric liquid crystal composition is injected into an accommodating space formed between the first substrate and the second substrate.

In Step S22, the cholesteric liquid crystal composition is irradiated with light of a predetermined wavelength to form a first cholesteric liquid crystal unit and a second cholesteric liquid crystal unit in the cholesteric liquid crystal layer.

The liquid crystal in the first cholesteric liquid crystal unit has an oblique heliconical structure with a first handedness, the liquid crystal in the second cholesteric liquid crystal unit has an oblique heliconical structure with a second handedness, and the first handedness is opposite to the second handedness.

Since cholesteric liquid crystals with two different handedness (e.g., left-handed and right-handed oblique heliconical cholesteric liquid crystals) are simultaneously formed in the cholesteric liquid crystal, both the left-handed reflected light and the right-handed reflected light may be simultaneously reflected, thereby significantly improving the reflection contrast of the oblique heliconical cholesteric liquid crystal.

FIG. 12 shows a schematic diagram of light irradiation in a preparation of step S22 according to some embodiments of the present disclosure.

Exemplarily, in some embodiments of the present disclosure, referring to FIG. 12, the forming the cholesteric liquid crystal layer 3 between the first substrate 1 and the second substrate 2 includes: injecting the cholesteric liquid crystal composition into the accommodating space 4 formed between the first substrate 1 and the second substrate 2; irradiating the cholesteric liquid crystal composition with a mask 10 and light 20 of a predetermined wavelength, where the handedness of the liquid crystal in the cholesteric liquid crystal layer not exposed to the irradiation of the light with the predetermined wavelength remains unchanged to form a first cholesteric liquid crystal unit 31, and the handedness of the liquid crystal in the cholesteric liquid crystal layer exposed to the irradiation of the light with the predetermined wavelength is inversed to form a second cholesteric liquid crystal unit 32. For example, the first cholesteric liquid crystal unit 31 may include the oblique heliconical cholesteric liquid crystal with a first handedness, and the second cholesteric liquid crystal unit 32 may include the oblique heliconical cholesteric liquid crystal with a second handedness.

By designing the light irradiation range, the cholesteric liquid crystal composition may be selectively covered by light irradiation. For example, through the mask design, the cholesteric liquid crystal composition in some regions of the display panel may be covered by light irradiation, while the cholesteric liquid crystal composition in other regions of the display panel is not covered by light irradiation due to being blocked by the light-shielding part of the mask, such that the cholesteric liquid crystal layer may simultaneously include a first cholesteric liquid crystal unit 31 with a first-handedness oblique heliconical structure and a second cholesteric liquid crystal unit 32 with a second-handedness oblique heliconical structure, for example, the cholesteric liquid crystal display panel may simultaneously include the left-handed cholesteric liquid crystal and the right-handed cholesteric liquid crystal.

For example, an electric field is applied to the cholesteric liquid crystal display panel, and the cholesteric liquid crystal display panel is irradiated by 450 nm blue light in the presence of the mask, such that the part of the cholesteric liquid crystal exposed to blue light undergoes an inversion of the oblique heliconical structure from right-handed to left-handed due to photosensitivity, while the unexposed part of the cholesteric liquid crystal remains in the right-handed oblique heliconical structure, and through the chiral cancellation mechanism and an HTP value test of an actual system, a system with precisely controlled left-handed and right-handed handedness, where helical twisting powers are equal in magnitude but opposite in handedness, may be achieved. When an electric field is applied again, the system simultaneously has left-handedness and right-handedness and may reflect the same reflection color, thereby significantly improving the reflection contrast of the oblique heliconical liquid crystal device.

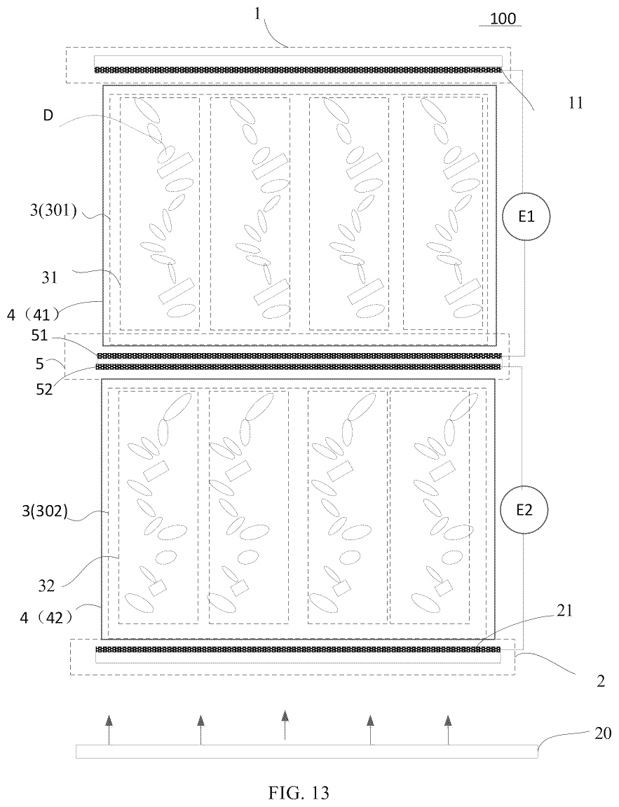

FIG. 13 shows a schematic diagram of light irradiation in a preparation of step S22 according to other embodiments of the present disclosure.

Exemplarily, in some embodiments of the present disclosure, referring to FIG. 13, the forming a cholesteric liquid crystal layer between the first substrate 1 and the second substrate 2 includes:

injecting the cholesteric liquid crystal composition into an accommodating space 41 formed between the first substrate 1 and the third substrate 5 to form a first sub-cholesteric liquid crystal layer 301;

injecting the cholesteric liquid crystal composition into an accommodating space 42 formed between the third substrate 5 and the second substrate 2 to form a second sub-cholesteric liquid crystal layer 302; and irradiating the second sub-cholesteric liquid crystal layer 302 with light of a predetermined wavelength, where the liquid crystal in the second sub-cholesteric liquid crystal layer is exposed to the irradiation of the light of the predetermined wavelength and undergoes handedness inversion to form a second cholesteric liquid crystal unit 32; the liquid crystal in the first sub-cholesteric liquid crystal layer 301 is not exposed to the irradiation of the light of the predetermined wavelength and remains handedness unchanged to form a first cholesteric liquid crystal unit 31.

By designing a display panel with a stacked cholesteric liquid crystal structure, one cholesteric liquid crystal layer is irradiated with light of a predetermined wavelength, while another cholesteric liquid crystal layer is not irradiated with light, such that the cholesteric liquid crystal layer may simultaneously include a first cholesteric liquid crystal unit with an oblique heliconical structure of a first handedness and a second cholesteric liquid crystal unit with an oblique heliconical structure of a second handedness, where the first handedness is opposite to the second handedness. For example, the first handedness may be left-handed, and the corresponding second handedness may be right-handed; alternatively, the first handedness may be right-handed, and the corresponding second handedness may be left-handed. That is, the cholesteric liquid crystal display panel may simultaneously include the left-handed cholesteric liquid crystal and the right-handed cholesteric liquid crystal.

Exemplarily, in some embodiments of the present disclosure, the stacked conductive liquid crystal cell is made of two conductive ITO-coated glass substrates subjected to reverse planar treatment and one double-sided conductive ITO-coated glass substrate subjected to reverse planar treatment. Specifically, the two conductive ITO-coated glass substrates subjected to reverse planar treatment are used to prepare an intermediate electrode so that upper and lower systems share the same intermediate electrode. The system is first poured into the stacked liquid crystal cell, and after applying an electric field to one layer of liquid crystal cell and irradiating the one layer of liquid crystal cell with 450 nm light until a stable state is reached, the liquid crystal cell in this layer is induced to have an oblique heliconical structure with a handedness opposite to the handedness of the other layer of liquid crystal system due to the presence of the chiral photosensitive molecular switch, such that the stacked liquid crystal cell may simultaneously reflect the left-handed reflected light and the right-handed reflected light, ultimately significantly improving the reflection contrast of the oblique heliconical liquid crystal device.

Both the mask design and the stacked liquid crystal structure design may achieve stable-state regulation of different regions in the oblique heliconical cholesteric liquid crystal, enabling the simultaneous presence of the left-handed oblique heliconical cholesteric liquid crystal and the right-handed oblique heliconical cholesteric liquid crystal in the liquid crystal, such that the liquid crystal cell may simultaneously reflect the left-handed reflected light and the right-handed reflected light, ultimately significantly improving the reflection contrast of the oblique heliconical liquid crystal device.

Although some embodiments of the overall technical concept of the present disclosure have been shown and described, those of ordinary skill in the art will understand that changes may be made to these embodiments without departing from the principles and spirit of the overall technical concept. The scope of the present disclosure is defined by the claims and their equivalents.

1. A chiral photosensitive molecular switch, wherein a chemical structure of the chiral photosensitive molecular switch is represented by the following general formula I: 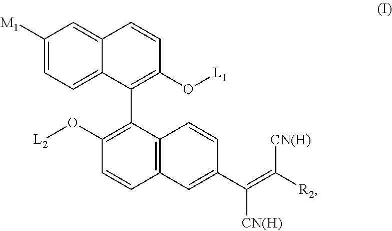
wherein M1 is selected from groups with structures as shown in 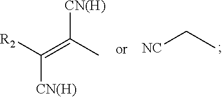
wherein groups R₁ and R₂ are the same or different, each comprising a benzene ring, biphenyl, an aromatic ring or an aromatic heterocyclic ring; and
wherein L₁ and L₂ each comprises a C₁-C₁₀ alkyl group.

2. The chiral photosensitive molecular switch according to claim 1, wherein a chemical structure of at least one of the groups R₁ and R₂ is represented by one of the following formulas:

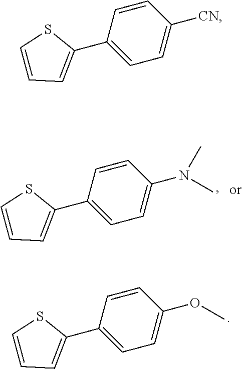

3. The chiral photosensitive molecular switch according to claim 1, wherein the chemical structure of the chiral photosensitive molecular switch is represented by one of the following formulas:

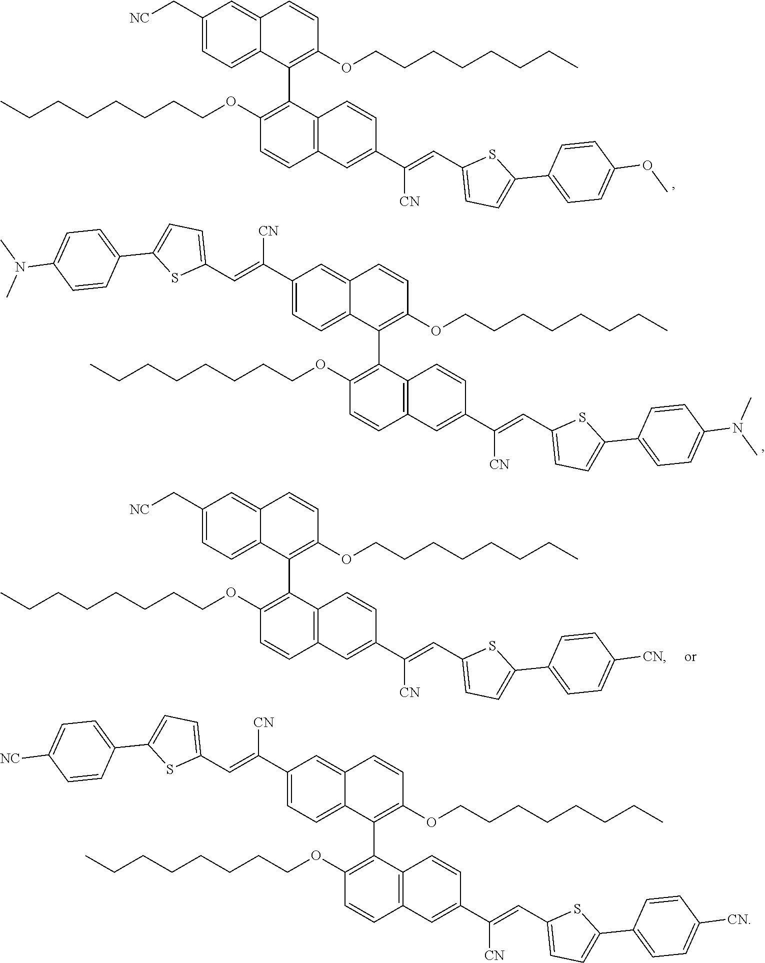

4. A cholesteric liquid crystal composition, comprising the chiral photosensitive molecular switch according to claim 1.

5. The cholesteric liquid crystal composition according to claim 4, further comprising a bent-core molecular mixture, a rod-like single crystal mixture and a non-photosensitive chiral dopant.

6. The cholesteric liquid crystal composition according to claim 5, wherein a proportion of the chiral photosensitive molecular switch is 0.5% to 10% by weight of the cholesteric liquid crystal composition.

7. The cholesteric liquid crystal composition according to claim 5, wherein a proportion of the non-photosensitive chiral dopant is 0.5% to 10% by weight of the cholesteric liquid crystal composition.

8. The cholesteric liquid crystal composition according to claim 5, wherein a proportion of the bent-core molecular mixture is 30% to 70% by weight of the cholesteric liquid crystal composition, or a proportion of the rod-like single crystal mixture is 30% to 70% by weight of the cholesteric liquid crystal composition.

9. The cholesteric liquid crystal composition according to claim 5,
wherein by weight of the cholesteric liquid crystal composition, a proportion of the bent-core molecular mixture is 30% to 70%, a proportion of the rod-like single crystal mixture is 30% to 70%, a proportion of the chiral photosensitive molecular switch is 5% to 10%, and a proportion of the non-photosensitive chiral dopant is 5% to 10%, and
wherein a sum of the proportion of the bent-core molecular mixture, the proportion of the rod-like single crystal mixture, the proportion of the chiral photosensitive molecular switch and the proportion of the non-photosensitive chiral dopant is 100%.

10. A cholesteric liquid crystal display panel, comprising:
a first substrate and a second substrate arranged opposite to each other; and
a cholesteric liquid crystal layer located between the first substrate and the second substrate,
wherein the cholesteric liquid crystal layer comprises a cholesteric liquid crystal composition, and the cholesteric liquid crystal composition comprises the chiral photosensitive molecular switch according to claim 1.

11. The display panel according to claim 10, wherein the cholesteric liquid crystal layer comprises a first cholesteric liquid crystal unit and a second cholesteric liquid crystal unit, a liquid crystal in the first cholesteric liquid crystal unit has an oblique heliconical structure with a first handedness, a liquid crystal in the second cholesteric liquid crystal unit has an oblique heliconical structure with a second handedness, and the first handedness is opposite to the second handedness.

12. The display panel according to claim 11, wherein an absolute value of a helical twisting power of the liquid crystal in the first cholesteric liquid crystal unit is substantially equal to an absolute value of a helical twisting power of the liquid crystal in the second cholesteric liquid crystal unit.

13. The display panel according to claim 10, further comprising: a first electrode located on the first substrate and a second electrode located on the second substrate;
wherein the cholesteric liquid crystal layer is configured such that: a wavelength corresponding to a reflection peak of the cholesteric liquid crystal layer changes in response to a change in a voltage applied between the first electrode and the second electrode.

14. The display panel according to claim 13, wherein the cholesteric liquid crystal layer is configured such that:
in response to a first voltage applied between the first electrode and the second electrode, the reflection peak of the cholesteric liquid crystal layer corresponds to a first wavelength;
in response to a second voltage applied between the first electrode and the second electrode, the reflection peak of the cholesteric liquid crystal layer corresponds to a second wavelength; and
in response to a third voltage applied between the first electrode and the second electrode, the reflection peak of the cholesteric liquid crystal layer corresponds to a third wavelength,
wherein the first voltage, the second voltage and the third voltage are different from each other, and the first wavelength, the second wavelength and the third wavelength correspond to visible light of different colors respectively;
wherein the cholesteric liquid crystal layer is configured such that: the wavelength corresponding to the reflection peak of the cholesteric liquid crystal layer increases in response to a decrease in the voltage applied between the first electrode and the second electrode;
wherein the first voltage is greater than the second voltage, and the second voltage is greater than the third voltage; and
wherein the first wavelength is less than the second wavelength, and the second wavelength is less than the third wavelength.

15-16. (canceled)

17. The display panel according to claim 11, wherein the first substrate and the second substrate are cell-assembled to form an accommodating space, the cholesteric liquid crystal layer is located in the accommodating space, and orthographic projections of the first cholesteric liquid crystal unit and the second cholesteric liquid crystal unit on the first substrate are distributed alternately.

18. The display panel according to claim 11, further comprising: a third substrate located between the first substrate and the second substrate;
wherein the cholesteric liquid crystal layer comprises: a first sub-cholesteric liquid crystal layer located between the first substrate and the third substrate, and a second sub-cholesteric liquid crystal layer located between the third substrate and the second substrate; and
wherein the first sub-cholesteric liquid crystal layer comprises the first cholesteric liquid crystal unit, and the second sub-cholesteric liquid crystal layer comprises the second cholesteric liquid crystal unit.

19. A method of preparing a cholesteric liquid crystal display panel, comprising:
providing a first substrate and a second substrate; and
forming a cholesteric liquid crystal layer between the first substrate and the second substrate,
wherein the cholesteric liquid crystal layer comprises a cholesteric liquid crystal composition, and the cholesteric liquid crystal composition comprises the chiral photosensitive molecular switch according to claim 1.

20. The method according to claim 19, wherein the forming a cholesteric liquid crystal layer between the first substrate and the second substrate comprises:
injecting the cholesteric liquid crystal composition into an accommodating space formed between the first substrate and the second substrate; and
irradiating the cholesteric liquid crystal composition with light of a predetermined wavelength to form a first cholesteric liquid crystal unit and a second cholesteric liquid crystal unit in the cholesteric liquid crystal layer,
wherein a liquid crystal in the first cholesteric liquid crystal unit has an oblique heliconical structure with a first handedness, a liquid crystal in the second cholesteric liquid crystal unit has an oblique heliconical structure with a second handedness, and the first handedness is opposite to the second handedness.

21. The method according to claim 20, wherein the forming a cholesteric liquid crystal layer between the first substrate and the second substrate comprises:
injecting the cholesteric liquid crystal composition into an accommodating space formed between the first substrate and the second substrate; and
irradiating the cholesteric liquid crystal composition with a mask and light of a predetermined wavelength, wherein a handedness of the liquid crystal in the cholesteric liquid crystal layer not exposed to the irradiation of the light of the predetermined wavelength remains unchanged to form a first cholesteric liquid crystal unit, and a handedness of the liquid crystal in the cholesteric liquid crystal layer exposed to the irradiation of the light of the predetermined wavelength is inversed to form a second cholesteric liquid crystal unit.

22. The method according to claim 20, wherein the forming a cholesteric liquid crystal layer between the first substrate and the second substrate comprises:
injecting the cholesteric liquid crystal composition into an accommodating space formed between the first substrate and a third substrate to form a first sub-cholesteric liquid crystal layer;
injecting the cholesteric liquid crystal composition into an accommodating space formed between the third substrate and the second substrate to form a second sub-cholesteric liquid crystal layer; and
irradiating the second sub-cholesteric liquid crystal layer with light of a predetermined wavelength, wherein a liquid crystal in the second sub-cholesteric liquid crystal layer is exposed to the irradiation of the light of the predetermined wavelength and undergoes handedness inversion to form a second cholesteric liquid crystal unit, and a liquid crystal in the first sub-cholesteric liquid crystal layer is not exposed to the irradiation of the light of the predetermined wavelength and remains handedness unchanged to form a first cholesteric liquid crystal unit.
